(19) United States
(12) Patent Application Publication
(10) Pub. No.: US 2026/0207811 A1
(43) Pub. Date: Jul. 23, 2026

(54) MATRICES FOR WOUND HEALING

(71) Applicant: Regenics AS, Oslo (NO)

(72) Inventors: Karin Gilljam, Oslo (NO); Henrik Lund, Oslo (NO)

(21) Appl. No.: 19/134,787

(22) PCT Filed: Nov. 30, 2023
(86) PCT No.: PCT/IB2023/062088

§ 371 Date: Jun. 2, 2025

Related U.S. Application Data
(60) Provisional application No. 63/429,705, filed on Dec. 2, 2022.

(51) Int. Cl. A61L 15/40; A61L 15/20; A61L 15/22; A61L 15/44; A61L 15/60; C08L 5/04; C08L 5/14

(52) U.S. Cl. CPC A61L 15/40; A61L 15/20; A61L 15/225; A61L 15/44; A61L 15/60; C08L 5/04; C08L 5/14; A61L 2300/30; A61L 2400/12

(57) ABSTRACT

The present invention provides articles for the improved healing of wounds, preferably burn wounds. In some preferred embodiments, the article is a matrix formed from a mixture of two or more polysaccharides, the matrix comprising an active ingredient which is an extract from differentiable cells.

### CROSS-REFERENCE TO RELATED APPLICATION

This application claims priority to U.S. Provisional Application No. 63/429,705, filed Dec. 2, 2022, the entire contents of each of which are incorporated herein by reference.

### FIELD OF THE INVENTION

The present invention provides articles for the improved healing of wounds, preferably burn wounds.

### BACKGROUND OF THE INVENTION

Burn injuries are traumatic, can be extremely painful and are associated with slow recovery. Burn injuries may also lead to severe mental and emotional distress caused by massive scarring. Healing of burn wounds follows the same steps as any wound, such as hemostasis, inflammation, proliferation, and remodeling. However, burn wounds differ from other types of wounds by the severity and duration of the inflammatory phase.1 In the inflammatory phase, neutrophils and monocytes are recruited to the wound, where they are responsible for removal of foreign materials and necrotic tissue. Upon activation, monocytes are transformed into macrophages (M0), which will be further transformed into pro-inflammatory (M1) or anti-inflammatory (M2) state macrophages. The pro-inflammatory M1 macrophages secrete chemokines such as interleukin (IL)-1β to sustain the inflammatory response, while anti-inflammatory M2 macrophages suppress inflammation and activates the proliferative phase (1,2). The inflammatory phase is a vital step in healing of all wounds, but in burn wounds, the pro-inflammatory M1 phenotype is predominant, causing an excessive and prolonged inflammatory response (3). This prolonged inflammation may cause secondary necrosis and progression of the burn wounds many days after the actual trauma (4).

Reactive oxygen species (ROS) are products of natural cellular respiration and act as second messengers in several cellular processes. In wound healing, ROS play a pivotal role in orchestration of several processes such as recruitment of lymphoid cells, effective tissue repair and angiogenesis.5 However, excessive ROS, called oxidative stress, cause damage to DNA, proteins and lipids and cause activation of pro-apoptotic proteins (6,7). Burn wounds are known to generate a massive production of ROS, which is one of the mechanisms responsible for the pathophysiological events observed after burn injury. Together with increased inflammation, increased ROS contributes to the secondary necrosis (6,7). Moreover, ROS may boost the inflammatory response, while the inflammatory response may boost the ROS levels, thus reinforcing each other. Increased inflammation and increased ROS not only delay wound healing, but also play a major role in the formation of hypertrophic scarring seen after severe burn wounds (8,9).

Current standard-of-care dressings for partial thickness burn wounds aim to cover and protect the wound surface from infection, maintain a moist environment, and reduce discomfort for the patient. In Norwegian hospitals, burn wounds are covered by the vaseline compress Jelonet® for the first day (10). Where the treatment does not call for skin transplantation, treatment of partial thickness burn wounds typically entail wound covering with Mepilex® Ag or Aquacel® Ag Burn (10). These dressings will contribute to moist healing, while reducing the risk of bacterial infection (11,12). Silver ions (Ag+) released from these dressing have antibacterial activity. However, studies have shown that high release of Ag+ are correlated with strong cytotoxicity, causing histological damage and delayed healing (12,13). Dressings with incorporated anti-inflammatory and antioxidant activity are being investigated, (14,15) however, to our knowledge, there are no such dressings in regular, i.e. non-research, clinical use.

What is needed in the art are new and effective treatments for burn wounds.

### SUMMARY OF THE INVENTION

The present invention provides articles for the improved healing of wounds, preferably burn wounds.

Accordingly, in some preferred embodiments, the present provides an article comprising a matrix formed from at least a first polysaccharide, said matrix further comprising a differentiable cell extract and wherein the first polysaccharide is from a source different from the differentiable cell extract.

In some preferred embodiments, the first polysaccharide is alginate. In some preferred embodiments, the weight/weight percent of the alginate in the article is from 1.0% to 10.0%. In some preferred embodiments, the weight/weight percent of the alginate in the article is from 1.0% to 3.0%. In some preferred embodiments, the weight/weight percent of the alginate in the article is from 1.5% to 2.5%. In some preferred embodiments, the weight/weight percent of the alginate in the article is from 1.85% to 2.15%. In some preferred embodiments, the weight/weight percent of the alginate in the article is from 1.9% to 2.1%.

In some preferred embodiments, the article further comprises a second polysaccharide from a source different the differentiable cell extract. In some preferred embodiments, the second polysaccharide is nanocellulose. In some preferred embodiments, the weight/weight percent of the nanocellulose in the article is from 1.0% to 10.0%. In some preferred embodiments, the weight/weight percent of the nanocellulose in the article is from 1.0% to 3.0%. In some preferred embodiments, the weight/weight percent of the nanocellulose in the article is from 1.2% to 2.2%. In some preferred embodiments, the weight/weight percent of the nanocellulose in the article is from 1.5% to 2.0%. In some preferred embodiments, the weight/weight percent of the nanocellulose in the article is from 1.55% to 1.95%. In some further preferred embodiments, the nanocellulose is supplemented with mannitol. In some preferred embodiments, the weight/weight percent of the mannitol used to supplement the nanocellulose is from 1.0% to 10.0%. In other preferred embodiments, the article comprises less than 0.5%, 0.1% or 0.01% w/w nanocellulose or is free from added nanocellulose.

In some preferred embodiments, the volume/weight percent of the differentiable cell extract in the article is from 5.0% to 20.0%. In some preferred embodiments, the volume/weight percent of the differentiable cell extract in the article is from 7.0% to 17.0%. In some preferred embodiments, the volume/weight percent of the differentiable cell extract in the article is from 9.0% to 15.0%. In some preferred embodiments, the volume/weight percent of the differentiable cell extract in the article is from 10.0% to 14.0%. In some preferred embodiments, the volume/weight percent of the differentiable cell extract in the article is from 11.0% to 13.0%.

In some preferred embodiments, the differentiable cell extract is a fish egg extract. In some preferred embodiments, the fish egg extract is a salmonid egg extract. In some preferred embodiments, the fish egg extract is a Salmo salar egg extract. In some preferred embodiments, the fish egg extract is an unfertilized egg extract. In some preferred embodiments, the fish egg extract is characterized is characterized in having one or more of properties (a) to (f): a) from 10 to 500 mg/ml protein and most preferably from 50 to 200 mg/ml protein, in an aqueous solution; b) from 0.1 to 10 mg/ml RNA; c) from 0.1 to 10 mg/ml DNA; d) from 0.1-10% lipids w/w e) an osmolarity of from 200 to 600 mOsm, most preferably from 330 to 440 mOsm; and f) a pH of from about 5.0 to 7.7. In some preferred embodiments, the fish egg extract has properties: (a) and (b); (a) and (c); (a) and (d); (a) and (e); (a) and (f); (a), (b), and (c); (a), (b), and (d); (a), (b), and (e); (a), (b), and (f); (a), (c), and (d); (a), (c), and (e); (a), (c), and (f); (a), (d), and (e); (a), (d), and (f); (a), (b), (c), and (d); (a), (b), (c), and (e); (a), (b), (c), and (f); (a), (c), (d), and (e); (a), (c), (d), and (f); (a), (c), (e), and (f); (a), (b), (c), (d) and (e); (a), (b), (c), (d) and (f); (a), (c), (d), (e) and (f); or (a), (b), (c), (d), (e), and (f).

In some preferred embodiments, the fish egg extract is a heat-treated fish egg extract. In some preferred embodiments, the heat-treated fish egg extract is prepared by heating the fish egg extract to from 90 to 100 degrees Celsius for from 1 to 30 minutes.

In some preferred embodiments, the first or the first and second polysaccharides are cross-linked.

In some preferred embodiments, the matrix is a gel matrix.

In some preferred embodiments, the matrix is formed in a grid pattern.

In some further preferred embodiments, the present invention provides an article comprising a matrix formed from alginate, said matrix further comprising a heat-treated Salmo salar egg extract, wherein the weight/weight percent of the alginate in the article is from 1.0% to 10.0% and the volume/weight percent of the heat-treated Salmo salar egg extract in the article is from 5.0% to 20.0%. In some preferred embodiments, the weight/weight percent of the alginate in the article is from 1.0% to 3.0%. In some preferred embodiments, the weight/weight percent of the alginate in the article is from 1.5% to 2.5%. In some preferred embodiments, the weight/weight percent of the alginate in the article is from 1.85% to 2.15%. In some preferred embodiments, the weight/weight percent of the alginate in the article is from 1.9% to 2.1%.

In some preferred embodiments, the article further comprises nanocellulose. In some preferred embodiments, the weight/weight percent of nanocellulose in the article is from 1.0% to 10.0%. In some preferred embodiments, the nanocellulose is supplemented with mannitol. In some preferred embodiments, the weight/weight percent of the mannitol used to supplement the nanocellulose is from 1.0% to 10.0%. In some preferred embodiments, the weight/weight percent of the nanocellulose in the article is from 1.0% to 3.0%. In some preferred embodiments, the weight/weight percent of the nanocellulose in the article is from 1.2% to 2.2%. In some preferred embodiments, the weight/weight percent of the nanocellulose in the article is from 1.5% to 2.0%. In some preferred embodiments, the weight/weight percent of the nanocellulose in the article is from 1.55% to 1.95%. In some preferred embodiments, the volume/weight percent of the differentiable cell extract in the article is from 7.0% to 17.0%. In some preferred embodiments, the volume/weight percent of the differentiable cell extract in the article is from 9.0% to 15.0%. In some preferred embodiments, the volume/weight percent of the differentiable cell extract in the article is from 10.0% to 14.0%. In some preferred embodiments, the volume/weight percent of the differentiable cell extract in the article is from 11.0% to 13.0%. In other preferred embodiments, the article comprises less than 0.5%, 0.1% or 0.01% w/w nanocellulose or is free from added nanocellulose.

In some preferred embodiments, the heat-treated Salmo salar egg extract is prepared from unfertilized eggs. In some preferred embodiments, the heat-treated Salmo salar egg extract is characterized is characterized in having one or more of properties (a) to (f): a) from 50 to 500 mg/ml protein and most preferably from 10 to 5000 mg/ml protein and most preferably from 50 to 200 mg/ml protein, in an aqueous solution; b) from 0.1 to 10 mg/ml RNA; c) from 0.1 to 10 mg/ml DNA; d) from 0.1-10% lipids w/w; e) an osmolarity of from 200 to 60 mOsm, most preferably from 330 to 440 mOsm; and f) a pH of from about 5.0 to 7.7. In some preferred embodiments, the fish egg extract has properties: (a) and (b); (a) and (c); (a) and (d); (a) and (e); (a) and (f); (a), (b), and (c); (a), (b), and (d); (a), (b), and (e); (a), (b), and (f); (a), (c), and (d); (a), (c), and (e); (a), (c), and (f); (a), (d), and (e); (a), (d), and (f); (a), (b), (c), and (d); (a), (b), (c), and (e); (a), (b), (c), and (f); (a), (c), (d), and (e); (a), (c), (d), and (f); (a), (c), (e), and (f); (a), (b), (c), (d) and (e); (a), (b), (c), (d) and (f); (a), (c), (d), (e) and (f); or (a), (b), (c), (d), (e), and (f). In some preferred embodiments, the heat-treated Salmo salar egg extract is prepared by heating the Salmo salar egg extract to from 90 to 100 degrees Celsius for from 1 to 30 minutes.

In some preferred embodiments, the matrix is a cross-linked gel matrix. In some preferred embodiments, the matrix is formed in a grid pattern.

In some preferred embodiments, the present invention provides methods of producing a wound healing article comprising: forming an aqueous mixture of at least a first polysaccharide and a fish egg extract; forming a matrix from the aqueous mixture to provide the wound healing article; and wherein the at least a first polysaccharide is from a source different from the fish egg extract.

In some preferred embodiments, the methods further comprise the step of cross linking the matrix to provide the wound healing article.

In some preferred embodiments, the first polysaccharide is alginate. In some preferred embodiments, the alginate is included in the mixture at a weight/weight percent of from 1.0% to 10.0%. In some preferred embodiments, the weight/weight percent of the alginate in the article is from 1.0% to 3.0%. In some preferred embodiments, the weight/weight percent of the alginate in the article is from 1.5% to 2.5%. In some preferred embodiments, the weight/weight percent of the alginate in the article is from 1.85% to 2.15%. In some preferred embodiments, the weight/weight percent of the alginate in the article is from 1.9% to 2.1%.

In some preferred embodiments, the methods further comprise including a second polysaccharide in the aqueous mixture, wherein the second polysaccharide is from a source different from the fish egg extract. In some preferred embodiments, the second polysaccharide is nanocellulose. In some preferred embodiments, the nanocellulose is included in the mixture at a weight/weight percent of from 1.0% to 10.0%. In some preferred embodiments, the weight/weight percent of the nanocellulose in the article is from 1.0% to 3.0%. In some preferred embodiments, the weight/weight percent of the nanocellulose in the article is from 1.2% to 2.2%. In some preferred embodiments, the weight/weight percent of the nanocellulose in the article is from 1.5% to 2.0%. In some preferred embodiments, the weight/weight percent of the nanocellulose in the article is from 1.55% to 1.95%. In other preferred embodiments, the methods utilize less than 0.5%, 0.1% or 0.01% w/w nanocellulose or do not comprise the addition of nanocellulose.

In some preferred embodiments, the fish egg extract is included in the mixture at a volume/weight percent of from 5.0% to 20.0%. In some preferred embodiments, the volume/weight percent of the differentiable cell extract in the article is from 7.0% to 17.0%. In some preferred embodiments, the volume/weight percent of the differentiable cell extract in the article is from 9.0% to 15.0%. In some preferred embodiments, the volume/weight percent of the differentiable cell extract in the article is from 10.0% to 14.0%. In some preferred embodiments, the volume/weight percent of the differentiable cell extract in the article is from 11.0% to 13.0%. In some preferred embodiments, the fish egg extract is from unfertilized fish eggs. In some preferred embodiments, the fish egg extract is a Salmo salar egg extract. In some preferred embodiments, the fish egg extract is a heat-treated fish egg extract. In some preferred embodiments, the heat-treated fish egg extract is prepared by heating the fish egg extract to a temperature of from 90 to 100 degrees Celsius for from 1 to 30 minutes. In some preferred embodiments, the heat-treated Salmo salar egg extract is characterized is characterized in having one or more of properties (a) to (f): a) from 10 to 500 mg/ml protein and most preferably from 50 to 200 mg/ml protein, in an aqueous solution; b) from 0.1 to 10 mg/ml RNA; c) from 0.1 to 10 mg/ml DNA; d) from 0.1-10% lipids w/w; e) an osmolarity of from 200 to 60 mOsm, most preferably from 330 to 440 mOsm; and f) a pH of from about 5.0 to 7.7. In some preferred embodiments, the fish egg extract has properties: (a) and (b); (a) and (c); (a) and (d); (a) and (e); (a) and (f); (a), (b), and (c); (a), (b), and (d); (a), (b), and (e); (a), (b), and (f); (a), (c), and (d); (a), (c), and (e); (a), (c), and (f); (a), (d), and (e); (a), (d), and (f); (a), (b), (c), and (d); (a), (b), (c), and (e); (a), (b), (c), and (f); (a), (c), (d), and (e); (a), (c), (d), and (f); (a), (c), (e), and (f); (a), (b), (c), (d) and (e); (a), (b), (c), (d) and (f); (a), (c), (d), (e) and (f); or (a), (b), (c), (d), (e), and (f).

In some preferred embodiments, the nanocellulose is supplemented with mannitol. In some preferred embodiments, the weight/weight percent of the mannitol used to supplement the nanocellulose is from 1.0% to 10.0%.

In some preferred embodiments, the aqueous mixture further comprises CaCl₂). In some preferred embodiments, the CaCl₂) is included in the aqueous mixture at a concentration of from 0.01 to 0.1 M.

In some preferred embodiments, the matrix is formed by printing the aqueous mixture onto a substrate.

In some preferred embodiments, the matrix is formed by molding the aqueous mixture.

In some preferred embodiments, the matrix is cross-linked by treating the matrix with a cross-linking solution comprising CaCl₂) at a concentration of from 0.01 to 0.1 M. In some preferred embodiments, the cross-linking solution further comprises a weight/weight percent of NaCl of from 0.5% to 1.5%. In some preferred embodiments, comprises the fish egg extract at a volume/weight percent of from 5.0% to 20.0%.

In some preferred embodiments, the matrix is a gel.

In some preferred embodiments, the present invention provides a matrix made by any of the foregoing methods.

In some preferred embodiments, the present invention provides an article or matrix as described above for use in treating a wound in a subject. In some preferred embodiments, the wound is a burn wound. In some preferred embodiments, the wound is a chronic wound. In some preferred embodiments, the article or solid matrix is topically applied to the wound.

In some preferred embodiments, the present invention provides a method of treating a wound in a subject in need thereof comprising applying an article or matrix as described above to the wound. In some preferred embodiments, the wound is a burn wound. In some preferred embodiments, the wound is a chronic wound.

In some preferred embodiments, the present invention provides for the use of an article or matrix as described above to reduce reactive oxygen species in a subject in need thereof. In some preferred embodiments, the article or matrix is applied at a site on the subject that exhibits or is at risk of inflammation. In some preferred embodiments, the subject has a wound and the matrix is applied to the wound. In some preferred embodiments, the subject has skin inflammation and the matrix is applied to the site of skin inflammation.

In some preferred embodiments, the present invention provides for the use of a fish egg extract or formulation thereof to reduce reactive oxygen species in a subject in need thereof, wherein the fish egg extract is characterized in having one or more of properties (a) to (f): a) from 50 to 500 mg/ml protein and most preferably from 10 to 5000 mg/ml protein and most preferably from 50 to 200 mg/ml protein, in an aqueous solution; b) from 0.1 to 10 mg/ml RNA; c) from 0.1 to 10 mg/ml DNA; d) from 0.1-10% lipids w/w e) an osmolarity of from 200 to 600 mOsm, most preferably from 330 to 440 mOsm; and f) a pH of from about 5.0 to 7.7. In some preferred embodiments, the extract is applied at a site on the subject that exhibits or is at risk of inflammation. In some preferred embodiments, the subject has a wound and the extract is applied to the wound. In some preferred embodiments, the subject has skin inflammation and the extract is applied to the site of skin inflammation. In some preferred embodiments, the article or matrix is applied at a site on the subject that exhibits or is at risk of inflammation. In some preferred embodiments, the fish egg extract has properties: (a) and (b); (a) and (c); (a) and (d); (a) and (e); (a) and (f); (a), (b), and (c); (a), (b), and (d); (a), (b), and (e); (a), (b), and (f); (a), (c), and (d); (a), (c), and (e); (a), (c), and (f); (a), (d), and (e); (a), (d), and (f); (a), (b), (c), and (d); (a), (b), (c), and (e); (a), (b), (c), and (f); (a), (c), (d), and (e); (a), (c), (d), and (f); (a), (c), (e), and (f); (a), (b), (c), (d) and (e); (a), (b), (c), (d) and (f); (a), (c), (d), (e) and (f); or (a), (b), (c), (d), (e), and (f). In some preferred embodiments, the fish egg extract is a heat-treated fish egg extract. In some preferred embodiments, the fish egg extract is a heat-treated fish egg extract.

### BRIEF DESCRIPTION OF THE FIGURES

FIG. 1. Burn procedure used on Göttingen minipig. (A) Illustration of burn wounds distribution on the back of the pig, duplicate burns, 8 burns on each side of the spine. The representative treatments were placed in such a way that all animals received all treatments, and the distribution close to the head and close to the tail was equal for all treatments. (B) Photo of custom-made burn device used to create partial thickness burns on the pigs. Aluminum rods (making duplicate burns) are directly connected to an aluminum block to ensure stable temperature. The block is attached to a heat-stable Plexiglas plate for easy handling. (C) Illustration of the dressings used on the minipigs. Orange circles illustrate the burn wounds. Because the Collex matrix, Collex matrix without HTX and Jelonet® are non-adherent dressings they were fixed with Mefix. Tegaderm was included to ensure that the dressings were kept in place the whole time during the experiment. (D) Schematic illustration of the timeline in the minipig burn study. Numbers on the arrow indicate days.

FIG. 2. The burns inflicted are partial thickness burn wounds and the Collex matrix shows signs of biocompatibility. (A) and (B) hematoxylin and eosin-stained biopsy slides of wounds at Day 4 (A) and 23 (B). (A) Burn depth as revealed by denaturation of dermal collagen (deeper eosinophilic staining). (B) Two representative wounds, as determined by evaluating pathologist, from each treatment at the end of the experiment: 1 and 2: Collex matrix, 3 and 4: Collex matrix without HTX, 5 and 6: Jelonet®. (C) Scores on reactivity of Collex matrix and Collex matrix without HTX compared to Jelonet®, determined by pathologist at Scantox A/S. Score 0.0-2.9: no reactivity, 3.0-8.9: slight rection, 9-15.0: moderate reaction, >15.1: severe reaction. Data presented is mean±SEM, N=3 (Day 4 and Day 10) and N=9 (Day 23). Blue bars represent reactivity in wounds treated with Collex matrix and green bars represent reactivity in wounds treated with Collex matrix without HTX.

FIG. 3. Collex matrix accelerates healing of partial thickness burn wounds. (A) Development of wound area from all wounds calculated from the planimetric drawings. Data presented are the mean values±SEM. Because wounds are terminated after biopsy at Day 4 and 10, number of wounds are not the same for all days: N=15 (Day 1 and 4), N=12 (Day 8 and 10) and N=9 (Day 12 to 23). Blue circles represent area of wounds treated with Collex matrix, green triangles represent area of wound treated with Collex matrix without HTX, and grey squares represent area of wound treated with Jelonet®. Grey star indicates P<0.05 between Collex and Jelonet®. Green star indicates P<0.05 between Collex matrix and Collex matrix without HTX. Significance is determined by unpaired T-test. (B) Photos of two representative burn wounds from each treatment, Day 19. Area of the wounds presented were closest to the mean area. Wound areas of the representative wounds: Collex matrix: 65 mm² and 52 mm² (mean: 58 mm²), Collex matrix without HTX: 63 mm² and 62 mm² (mean: 62 mm²), and Jelonet®: 72 mm² and 81 mm² (mean: 79 mm²).

FIG. 4. Burn wounds treated with Collex matrix have reduced inflammation. Inflammation scoring of the (A) wound edge and (B) surrounding skin based on macroscopic evaluation by Scantox A/S. (A and B) Grading system: 0: not present, 1: minimal, 2: slight, 3: moderate, 4: marked. Because wounds are terminated after biopsy at Day 4 and 10, number of wounds are not the same for all days: N=15 (Day 4) and N=12 (Day 6 and 8). Data shown is mean values±SEM. P values are determined by unpaired T-test. (C) Photos of two representative burn wounds from each treatment, Day 4. The wounds are the same as shown in FIG. 3B.

FIG. 5. In vitro studies show anti-inflammatory and antioxidant effect of the Collex matrix and HTX on M1-polarized macrophages. (A) Schematic illustration of method used for differentiation of THP-1 monocytes into macrophages, M0 and M1. (B) IL-1β secreted from macrophages activated according to (A), detected by ELISA assay. The data presented is normalized against the level secreted from cells treated with PMA, IFNγ and LPS (M1+Medium). (C) Cell death as determined by PI-positive cells. Cells are from the same experiment as (B). (D) ROS levels determined by CellROX measured on Flow cytometry. The data presented is normalized against the ROS level in cells treated with PMA, IFNγ and LPS (M1+Medium). Cells are from the same experiment as (B and C). (B, C and D) Data presented is mean values±SD, N=2 (PMA treatment only, M0) and N=3 (M1). P values are determined by unpaired T-test.

FIG. 6. Model describing the suggested mode of action for the Collex matrix. Burn wounds are heavily inflamed and have abundant secretion of pro-inflammatory IL-1β and excessive generation of ROS. Our data suggest that the Collex matrix (containing HTX) dampens the inflammatory response in vivo, possibly by reducing the level of the pro-inflammatory cytokine IL-1β and reducing the level of ROS. Both in the inflammatory and the proceeding proliferative phase of wound healing, Collex matrix appears to accelerate the wound closure by ensuring a moist, protected environment.

FIG. 7. Protein release from the Collex matrix in 1 ml DMEM supplemented with 10% FBS and 1% Pen/Strep determined by absorbance at 280 nm. The patches were incubated at 37° C., 5% CO2, humidified atmosphere during the analysis, N=5 presented as a mean±SD.

FIG. 8. Procollagen-1 alpha 1 ELISA assay results normalized to medium control. Data presented as mean with SD. ** indicates p<0.01. Collex 4W 20° C. (N=4) and Collex 4W 4° C. (N=2) was stored at room temperature and fridge for 4 weeks, respectively.

FIG. 9. Schematic drawing of the print grid design for an article of the instant invention.

FIG. 10. (a) Viscometry conducted on the bioinks, here shown with respect to viscosity (Pa s) vs. shear rate (s⁻¹), and (b) shear stress (Pa) vs. shear rate (s⁻¹). Mean values presented with SD (n=3).

FIG. 11. Frequency sweep describing the viscoelastic properties of the ink in terms of storage

modulus, G′ (Pa), in relation to angular frequency, ω (rad s⁻¹). Mean values presented with SD (n=3).

FIG. 12. Schematic CAD drawing of the continuous interlaced grid design (a), and an ongoing print of the second layer using the R4 ink (b). The dimensions of the CAD grid were 16 mm×16 mm 1.37 mm with a pore size of 1.4 mm×1.4 mm.

FIG. 13. Release from Collex matrix: Release in DMEM supplemented with 10% FBS and 1% Pen/Strep, incubated at room temperature. a) Collex matrix molded without nanocellulose (unpublished), b) Collex 3D printed matrix with nanocellulose.

FIG. 14. Procollagen-1 ELISA results: a) Collex matrix molded without nanocellulose, b) Collex matrix 3D printed with nanocellulose.

FIG. 15. ROS detection by CellROX deep red, 2 hours, 24 hours and 72 hours after HTX treatment of the fibroblast cell line HS707. The cells were starved (1% instead of 10% serum) for 24 hours prior to HTX treatment.

FIG. 16. ROS detection by CellROX deep red, 2 hours, 24 hours and 72 hours after HTX treatment of the keratinocyte cell line HaCaT. The cells were starved (1% instead of 10% serum) for 24 hours prior to HTX treatment.

### DEFINITIONS

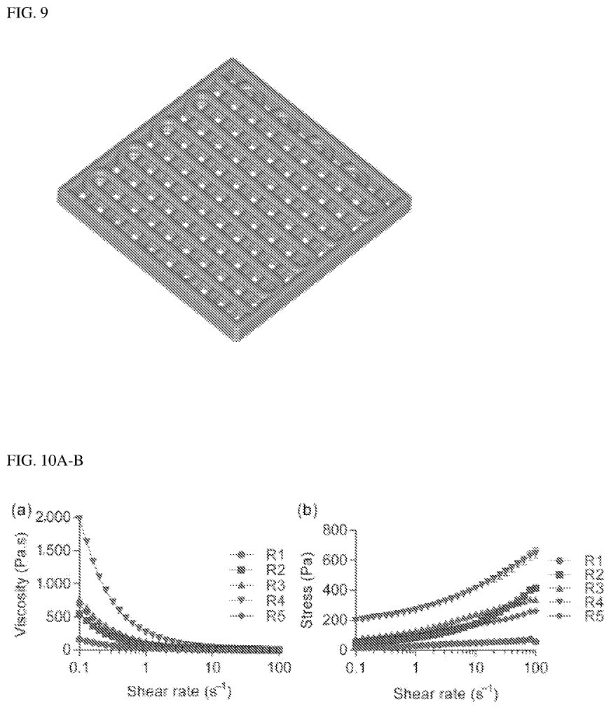

As used herein, the term “matrix” when used in reference to an article of the present invention refers to a material in which an agent (e.g., HTX; heat-treated egg extract) is incorporated into. The matrix will be understood to have a length, breadth, and depth and may assume a variety of shapes and patterns, including, but not limited to, circular, rectangular, triangular or square sheets, as well as grid and other patterns (see, e.g., FIG. 9).

As used herein, the term “HTX” refers to a heated treated extract of salmon eggs (See, e.g., PCT/IB2013/003177 and as described elsewhere herein).

As used herein, “cell” means the smallest structural unit of living matter capable of functioning autonomously, consisting of one or more nuclei, cytoplasm, and various organelles, all surrounded by a semipermeable membrane. Cells include all somatic cells obtained or derived from a living or deceased animal body at any stage of development as well as germ cells, including sperm and eggs (animal reproductive body consisting of an ovum or embryo together with nutritive and protective envelopes). Included are both general categories of cells: prokaryotes and eukaryotes. The cells contemplated for use in this invention include all types of cells from all organisms in all kingdoms: plants, animals, protists, fungi, archaebacteria and eubacteria. Stem cells are cells capable, by successive divisions, of producing specialized cells on many different levels. For example, hematopoietic stem cells produce both red blood cells and white blood cells. From conception until death, humans contain stem cells, but in adults their power to differentiate is reduced.

As used herein, the term “differentiation” related to cells means the process by which cells becomes structurally and functionally specialized, which is a progressive restriction of the developmental potential and increasing specialization of function which takes place during the development of the embryo and leads to the formation of specialized cells, tissues, and organs.

The term “dedifferentiation” related to cells means the reverse process of differentiation, where cells become less structurally and functionally specialized, which increases the developmental potential of the cell.

“Differentiable” means the ability of a cell to differentiate into a desired cell type. As used herein, the term “differentiates” means specialization (differentiation) or return to a more primitive cell type; dedifferentiation).

An “extract” as used in the context of “cell extract” and “egg extract” in this invention means a preparation of any type of cell as defined above obtained by chemical or mechanical action, as by pressure, distillation, evaporation etc. Extracts can include all or any single component or combination of components of the cells, including concentrated preparations of the active components. Such components of the extracts include but are not limited to RNA, DNA, micro RNA, lipids, free amino acids, all amino acid base structures including peptides and proteins, carbohydrates, minerals or combinations thereof. Extracts contemplated by this invention include but are not limited to extracts of fish eggs, urchin eggs, frog eggs, adult stem cells, plant seeds and plant stem cells.

The term “manage” when used in connection with a disease or condition means to provide beneficial effects to a subject being administered with a prophylactic or therapeutic agent, which does not result in a cure of the disease. In certain embodiments, a subject is administered with one or more prophylactic or therapeutic agents to manage a disease so as to prevent the progression or worsening of the disease.

As used herein, the terms “prevent” and “preventing” include the prevention of the recurrence, spread or onset. It is not intended that the present invention be limited to complete prevention. In some embodiments, the onset is delayed, or the severity of the disease is reduced.

As used herein, the terms “treat” and “treating” are not limited to the case where the subject (e.g. patient) is cured and the disease is eradicated. Rather, the present invention also contemplates treatment that merely reduces symptoms, and/or delays disease progression.

### DETAILED DESCRIPTION OF THE INVENTION

The present invention provides articles for the improved healing of wounds, preferably burn wounds. In some preferred embodiments, the article is a matrix formed from a mixture of two or more polysaccharides, the matrix comprising an active ingredient which is an extract from differentiable cells.

In some particularly preferred embodiments, the articles of the present invention are utilized to treat burn wounds. Partial thickness thermal burn wounds are characterized by prolonged inflammatory response, oxidative stress, tissue damage, and secondary necrosis. An optimal dressing for burn wounds can therefore reduce inflammation and oxidative stress while it provides a moist and absorbent cover, protecting the wound. In some preferred embodiments, the articles of the present invention comprise an extract from unfertilized salmon roe containing components with potential anti-inflammatory and antioxidative properties, called HTX. See, e.g., PCT Applications PCT/IB2011/001488 and PCT/IB2013/003177 which are both incorporated by reference herein their entirety. In the articles of the present invention, HTX has been combined with alginate from brown algae and nanocellulose from tunicates, and 3D printed into an all-marine hydrogel wound dressing, which is referred to herein as the Collex matrix. Thus, the present invention provides a technical solution for effective delivery of HTX as an active ingredient to a wound, for example a partial thickness burn wound.

Data provided herein describes testing of the Collex matrix on partial thickness burn wounds in Göttingen minipigs. The Collex matrix was compared to the vaseline compress Jelonet®, and a variant of the Collex matrix without HTX. It was found that dermal treatment of burn wounds with the Collex matrix resulted in accelerated healing compared to wounds treated with Jelonet®. Compared to the Collex matrix without HTX, the Collex matrix improved healing in the first week after trauma where secondary necrosis was pronounced. Notably, the Collex matrix reduced the inflammatory response in the early post-injury phase. The anti-inflammatory response of the Collex matrix was investigated in more detail on activated M1 macrophages. It was further found that the Collex matrix, as well as HTX alone, significantly reduced secretion of pro-inflammatory interleukin-1β as well as the intracellular level of oxidative stress. The results from this study suggest the Collex matrix is a potent dressing for treatment of burn wounds, with the anti-inflammatory effect of HTX beneficial in the initial phase, and the moist qualities of the hydrogel being favorable both in the initial and the proceeding proliferative phase of the wound healing.

Accordingly, in some embodiments, the present invention provides the present invention provides matrices, most preferably hydrogels, for delivery of an extract of differentiable cells (e.g., HTX) to a wound. In some preferred embodiments, the differentiable cell extract component provided in the matrix is characterized in having one or more of properties (a) to (f):
a) from 10 to 500 mg/ml protein and most preferably from 50 to 200 mg/ml protein, in an aqueous solution;
b) from 0.1 to 10 mg/ml RNA;
c) from 0.1 to 10 mg/ml DNA;
d) from 0.1-10% lipids w/w
e) an osmolarity of from 200 to 60 mOsm, most preferably from 330 to 440 mOsm;
f) a pH of from about 5.0 to 7.7.

In some preferred embodiments, the egg cellular extract is characterized in having two or more of properties (a) to (f). In some preferred embodiments, the differentiable cell extract is characterized in having three or more of properties (a) to (f). In some preferred embodiments, the differentiable cell extract is characterized in having four or more of properties (a) to (f). In some preferred embodiments, the differentiable cell extract is characterized in having five or more of properties (a) to (f). In some preferred embodiments, the differentiable cell extract is characterized in having all six of properties (a) to (f). In some preferred embodiments, the differentiable cell extract is characterized in having properties (a) and (b). In some preferred embodiments, the differentiable cell extract is characterized in having properties (a) and (c). In some preferred embodiments, the differentiable cell extract is characterized in having properties (a) and (d). In some preferred embodiments, the differentiable cell extract is characterized in having properties (a) and (e). In some preferred embodiments, the differentiable cell extract is characterized in having properties (a) and (f). In some preferred embodiments, the differentiable cell extract is characterized in having properties (a), (b), and (c). In some preferred embodiments, the differentiable cell extract is characterized in having properties (a), (b), and (d). In some preferred embodiments, the differentiable cell extract is characterized in having properties (a), (b), and (e). In some preferred embodiments, the differentiable cell extract is characterized in having properties (a), (b), and (f). In some preferred embodiments, the differentiable cell is characterized in having properties (a), (c), and (d). In some preferred embodiments, the differentiable cell extract is characterized in having properties (a), (c), and (e). In some preferred embodiments, the differentiable cell extract is characterized in having properties (a), (c), and (f). In some preferred embodiments, the differentiable cell extract is characterized in having properties (a), (d), and (e). In some preferred embodiments, the differentiable cell extract is characterized in having properties (a), (d), and (f). In some preferred embodiments, the differentiable cell extract is characterized in having properties (a), (b), (c), and (d). In some preferred embodiments, the differentiable cell extract is characterized in having properties (a), (b), (c), and (e). In some preferred embodiments, the differentiable cell extract is characterized in having properties (a), (b), (c), and (f). In some preferred embodiments, the differentiable cell extract is characterized in having properties (a), (c), (d), and (e). In some preferred embodiments, the differentiable cell extract is characterized in having properties (a), (c), (d), and (f). In some preferred embodiments, the differentiable cell extract is characterized in having properties (a), (c), (e), and (f). In some preferred embodiments, the differentiable cell extract is characterized in having properties (a), (b), (c), (d) and (e). In some preferred embodiments, the differentiable cell extract is characterized in having properties (a), (b), (c), (d) and (f). In some preferred embodiments, the differentiable cell extract is characterized in having properties (a), (c), (d), (e) and (f). In some preferred embodiments, the differentiable cell extract is characterized in having properties (a), (b), (c), (d), (e), and (f).

In some embodiments, the differentiable cell extract is selected from the group consisting of an extract of an activated fish egg cellular extract and an unactivated fish egg cellular extract. In some embodiments, the fish egg cellular extract is from a fertilized egg. In some embodiments, the fish egg cellular extract is from an unfertilized egg. In some embodiments, the cellular extract is heat treated by heating the extract to greater than 80 C, 90 C, 95 C or 100 C. In some embodiments, the heat treatment is from about 1 minute to about 30 minutes.

As just described, the compositions of the present invention utilize cell, egg and embryo extracts from vertebrates, including but not limited to Superclass Gnathostomata (jawed vertebrates), Euteleostomi (bony vertebrates), Class Actinopterygii (ray-finned fishes), Class Sarcopterygii (lobe-finned fishes and terrestrial vertebrates), Tetrapoda (tetrapods), Amniota (amniotes), Synapsida (synapsids), Class Mammalia (mammals), Early Therapsida (early therapsids), Class Reptilia (reptiles), Anapsida (tortoises and turtles), Order Testudines (tortoises and turtles), Diapsida (birds, crocodiles, lizards, snakes, and relatives), Archosauria (birds and crocodiles), Order Crocodilia (caimans, crocodiles, and relatives), Lepidosauria (amphisbaenians, lizards, snakes, and tuataras), Order Rhynchocephalia (tuataras), Order Squamata (amphisbaenians, lizards, and snakes), Class Amphibia (amphibians), Subclass Dipnoi (lungfishes), Actinistia, Order Coelacanthiformes (coelacanths), Class Chondrichthyes (rays, sharks, and relatives), Placodermi (armored fishes and placoderms), Class Cephalaspidomorphi, more preferably fish, shrimp, sea urchin or amphibian eggs or embryos. In some embodiments, unfertilized but activated fish, shrimp, sea urchin or amphibian eggs are used. The present invention is not limited to the use of any particular types of eggs. Indeed, the use of a variety of eggs is contemplated, including, but not limited to eggs from Xenopus, shrimp, sea urchin, salmon, trout or zebrafish. In some embodiments, eggs are collected from mature females and spontaneously activate upon contact with water. In further embodiments, the eggs are washed in Ringer's saline. In some embodiments, the eggs are not from an avian species. In some particularly preferred embodiments, the eggs are from a salmonid species. In some especially preferred embodiments, the salmonid is Salmo salar.

Extracts of the present invention are prepared from any of the sources described herein. In some embodiments, the extracts are cellular extracts. Cellular extracts of the present invention are preferably compositions of disrupted cells such as eggs. The cells may be disrupted by a variety of methods, including, but not limited to, mechanical shearing or blending, sonication, or osmotic lysis. In some embodiments, the extracts comprise less than about 1% and preferably less than 0.1% cholesterol or ovalbumin. Accordingly, in some embodiments, the cellular extract comprises carbohydrates, proteins, glycosylated or otherwise modified proteins, peptides, amino acids, RNA (mRNA, sRNA, miRNA, rRNA), DNA, water etc., and combinations thereof. In some embodiments, the cellular extracts can comprise small amounts of lipids naturally associated with the cells, as well as nuclear components such as chromosomes, nucleic acids, and nuclear proteins. In some embodiments, the cellular extract is preferably a cytoplasmic extract or fraction prepared by removing nuclear, cell membrane and other water insoluble materials naturally associated with the cells. In some embodiments, these components are removed by centrifugation or fractionation of the disrupted cells. In some embodiments, the cellular extract is preferably an aqueous extract or fraction comprising water soluble cellular components such as proteins, mRNA, and carbohydrates.

A variety of methods may be used to prepare extracts, including those described in the examples below. In some embodiments, eggs are placed “dry” in a glass 15 ml centrifuge tube, and crushed by sedimentation at 15,000 g for 15 min. This produces three layers: a lipid top fraction, which is collected, aliquoted and frozen; a middle cellular or cytoplasmic fraction, which is also collected, aliquoted and frozen; and a pellet fraction, which is discarded. In some embodiments, the cellular fraction or extract primarily comprises contents of the cytoplasm. The cellular fraction is used as extract. In some embodiments, the cellular fraction may be used in combination with a lipid fraction. The cytoplasmic fraction may be cleared further by sedimentation at 50,000, 100,000 or 200,000 g to yield a further cellular extract which is primarily a water soluble extract fraction. Regardless of the fraction used, the extract can be diluted to about 300 mOsm with cell lysis buffer (see above), if necessary. Accordingly, in some preferred embodiment's, a water soluble extract prepared from eggs or embryos is utilized.

In other embodiments, the eggs are suspended in 0.5 volume of cell lysis buffer and sonicated on ice until all eggs are lysed. The particulate material is sedimented at 15,000 g for 15 min at 4° C. The supernatant constitutes the extract. As above, osmolarity can be adjusted to 300 mOsm if needed. The extract can also be cleared as above.

In still other embodiments, the eggs are suspended in cell lysis buffer as in method 2. Eggs are lysed by Dounce homogenization using a glass mortar and pestle (Kontes, type A or B). The lysate is sedimented and treated as described above.

In some embodiments, the homogenates and extracts may be stabilized by the addition of one or more stabilizing agents, such as a lipid stabilizing agent, or by packaging in a package designed to prevent oxidation. In some embodiments, antioxidants such as vitamin E are added to the extract to reduce rate of lipid oxidation. In some embodiments, the extracts are packaged in a container under an inert atmosphere. In some embodiments, the extract is packaged to reduce rate of lipid oxidation in air-free containers such as aluminum coated bags (less than 10 kg per bag for efficient removal of oxygen), or containers filled with nitrogen to remove oxygen. In other embodiments, the extracts are packaged in vacuum packed containers with a pump delivery system.

In some embodiments, the present invention provides powders prepared from the cellular extracts described above. In some embodiments, the cellular extracts used in the production of the powders are prepared from salmonid eggs. In some embodiments, the cellular extracts used in the production of the powders are prepared from salmon or trout eggs. In some embodiments, the powders are biologically active. In some preferred embodiments, the powders are freeze-dried. In some embodiments, the powders have less than about 10% moisture and most preferably less than about 5% moisture; protein in a concentration of from about 500 to about 800 mg/g powder, preferably from about 600 to about 700 mg/g powder, most preferably about 640 mg/g powder; DNA in a concentration of from about 1 to about 50 μl/mg powder, preferably from about 5 to about 25 μl/mg powder, and most preferably about 16 μl/mg powder; total RNA (e.g., including mRNA, rRNA, and microRNA) in a concentration of from about 1 to about 50 μl/mg powder, preferably from about 5 to about 20 μl/mg powder, and most preferably about 12 μl/mg powder; and lipids in a concentration of from about 100 to about 200 mg/g powder, most preferably about 150 mg/g powder. The powders may preferably be used to make the formulations described herein as an alternative to the non-powdered cellular extracts.

In some particularly preferred embodiments, the cell extracts described above are incorporated into a matrix formed from one or more polysaccharides or a mixture of polysaccharides. Accordingly, in some preferred embodiments, the present invention provides methods of producing a wound healing article comprising: forming an aqueous mixture of at least a first polysaccharide and a differentiable cell extract; forming a matrix from the aqueous mixture; and optionally cross-linking matrix to provide a wound healing article. In some preferred embodiments, a second polysaccharide is included in the aqueous mixture.

In some particularly preferred embodiments, the differentiable cell extract is a fish egg extract. In some preferred embodiments, the fish egg extract is a salmonid egg extract. In some more preferred embodiments, the salmonid egg extract is a Salmo salar egg extract. In some preferred embodiments, the fish egg extracts, such as Salmo salar egg extracts, are prepared from unfertilized eggs. In some preferred embodiments, the cellular extract is included in the mixture at a volume/weight percent of from 5.0% to 20.0%, where volume is the volume of the cellular extract in milliliters and weight is the weight of the remainder of the components of the mixture in grams. In some preferred embodiments, the cellular extract is included in the mixture at a volume/weight percent of from 8.0% to 16.0%. In some preferred embodiments, the cellular extract is included in the mixture at a volume/weight percent of from 10.0% to 14.0%. In some embodiments, the cellular extract may be reconstituted from a powder prepared as described above. In some preferred embodiments, the fish egg extract is a heat-treated fish egg extract. In some preferred embodiments, the heat-treated fish egg extract is prepared by heating the fish egg extract to a temperature of from 90 to 100 degrees Celsius for from 1 to 30 minutes. In some embodiments, the cell extract, which may preferably be a heat-treated Salmo salar egg extract, is characterized is characterized in having one or more of properties (a) to (f):
a) from 10 to 500 mg/ml protein and most preferably from 50 to 200 mg/ml protein, in an aqueous solution;
b) from 0.1 to 10 mg/ml RNA;
c) from 0.1 to 10 mg/ml DNA;
d) from 0.1-10% lipids w/w
e) an osmolarity of from 200 to 60 mOsm, most preferably from 330 to 440 mOsm;
f) a pH of from about 5.0 to 7.7.
   In some preferred embodiments, the fish egg extract has properties: (a) and (b); (a) and (c); (a) and (d); (a) and (e); (a) and (f); (a), (b), and (c); (a), (b), and (d); (a), (b), and (e); (a), (b), and (f); (a), (c), and (d); (a), (c), and (e); (a), (c), and (f); (a), (d), and (e); (a), (d), and (f); (a), (b), (c), and (d); (a), (b), (c), and (e); (a), (b), (c), and (f); (a), (c), (d), and (e); (a), (c), (d), and (f); (a), (c), (e), and (f); (a), (b), (c), (d) and (e); (a), (b), (c), (d) and (f); (a), (c), (d), (e) and (f); or (a), (b), (c), (d), (e), and (f).

In some particularly preferred embodiments, the at least two polysaccharides are from a source different from the source of the differentiable cell extract, for example, from a different source than Salmo salar. In some preferred embodiments, the polysaccharides are from a marine source that is different than the source of the cell extract.

In some particularly preferred embodiments, the first polysaccharide is alginate. In some preferred embodiments, the alginate is included in the aqueous mixture at a weight/weight percent of from 1.0% to 10.0%, and most preferably from 1.0% to 3.0%, where weight/weight is the weight of the alginate per the total weight of the aqueous mixture.

In some particularly preferred embodiments, the second polysaccharide is nanocellulose. In some preferred embodiments, the nanocellulose is included in the aqueous mixture at a weight/weight percent of from 1.0% to 10.0%, and most preferably from 1.0% to 3.0%, where weight/weight is the weight of the nanocellulose per the total weight of the aqueous mixture.

In some preferred embodiments, the nanocellulose used to form the matrix further comprises mannitol. In some preferred embodiments, the nanocellulose is optionally supplemented with mannitol at weight percent of from 1.0% to 10.0% and most preferably from 2.0% to 7.0% where weight/weight is the weight of the mannitol per the total weight of the nanocellulose.

In some preferred embodiments, the aqueous mixture used to form the matrix further comprises CaCl₂). In some preferred embodiments, the CaCl₂) is included in the aqueous mixture at a concentration of from 0.01 to 0.1 M.

The present invention is not limited to any particular method of forming the matrix from the aqueous mixture. In some preferred embodiments, the matrix is formed by printing the aqueous mixture onto a substrate. In other preferred embodiments, the matrix is formed by molding the aqueous mixture. Suitable molds include release molds formed from, for example, polydimethylsiloxane (PDMS) or a release-coated polymer. Suitable release coatings are known in the art in the art and include silicone release coatings.

The present invention is not limited to any particular method of cross-linking the polymers used to form the matrix. In some embodiments, the matrix is cross-linked by treating the matrix with a cross-linking solution comprising CaCl₂) at a concentration of from 0.01 to 0.1 M. In some preferred embodiments, the cross-linking solution further comprises a weight/weight percent of NaCl of from 0.5% to 1.5%. In some preferred embodiments, the cross-linking solution further comprises the fish egg extract at a volume/weight percent of from 5.0% to 20.0%, and most preferably from 5.0% to 17.0%. In some preferred embodiments, the fish egg extract is included in the cross-linking solution at a volume/weight percent of from 8.0% to 16.0%. In some preferred embodiments, the cellular extract is included in the cross-linking solution at a volume/weight percent of from 10.0% to 14.0%.

In some preferred embodiments, the matrix is a gel.

In some preferred embodiments, the matrix is formed into a grid pattern.

In some preferred embodiments, the present invention provides a matrix made by the methods described above. According, in some embodiments, the present invention provides an article comprising a matrix (e.g., a gel matrix and most preferably a hydrogel matrix) formed from at least a first polysaccharide, said matrix further comprising a differentiable cell extract and wherein the first polysaccharide is from a source different from the differentiable cell extract. In some preferred embodiments, a second polysaccharide is included in the aqueous mixture, wherein the first polysaccharide is from a source different from the differentiable cell extract.

In some particularly preferred embodiments, the differentiable cell extract is a fish egg extract. In some preferred embodiments, the fish egg extract is a salmonid egg extract. In some more preferred embodiments, the salmonid egg extract is a Salmo salar egg extract. In some preferred embodiments, the fish egg extracts, such as Salmo salar egg extracts, are prepared from unfertilized eggs. In some preferred embodiments, the cellular extract is included in the mixture at a volume/weight percent of from 5.0% to 20.0%. In some preferred embodiments, the cellular extract is included in the mixture at a volume/weight percent of from 8.0% to 16.0%. In some preferred embodiments, the cellular extract is included in the mixture at a volume/weight percent of from 10.0% to 14.0%. In some preferred embodiments, the fish egg extract is a heat-treated fish egg extract. In some preferred embodiments, the heat-treated fish egg extract is prepared by heating the fish egg extract to a temperature of from 90 to 100 degrees Celsius for from 1 to 30 minutes. In some embodiments, the cell extract, which may preferably be a heat-treated Salmo salar egg extract, is characterized is characterized in having one or more of properties (a) to (f):
a) from 10 to 500 mg/ml protein and most preferably from 50 to 200 mg/ml protein, in an aqueous solution;
b) from 0.1 to 10 mg/ml RNA;
c) from 0.1 to 10 mg/ml DNA;
d) from 0.1-10% lipids w/w
e) an osmolarity of from 200 to 60 mOsm, most preferably from 330 to 440 mOsm;
f) a pH of from about 5.0 to 7.7.
   In some preferred embodiments, the fish egg extract has properties: (a) and (b); (a) and (c); (a) and (d); (a) and (e); (a) and (f); (a), (b), and (c); (a), (b), and (d); (a), (b), and (e); (a), (b), and (f); (a), (c), and (d); (a), (c), and (e); (a), (c), and (f); (a), (d), and (e); (a), (d), and (f); (a), (b), (c), and (d); (a), (b), (c), and (e); (a), (b), (c), and (f); (a), (c), (d), and (e); (a), (c), (d), and (f); (a), (c), (e), and (f); (a), (b), (c), (d) and (e); (a), (b), (c), (d) and (f); (a), (c), (d), (e) and (f); or (a), (b), (c), (d), (e), and (f).

In some particularly preferred embodiments, the polysaccharides (e.g., the first polysaccharide and/or the second polysaccharide) are from a source different from the source of the differentiable cell extract, for example, from a different source than Salmo salar. In some preferred embodiments, the polysaccharides are from a marine source that is different than the source of the cell extract.

In some particularly preferred embodiments, the first polysaccharide is alginate. In some preferred embodiments, the alginate is included in the aqueous mixture at a weight/weight percent of from 1.0% to 10.0% and most preferably from 1.0% to 3.0%.

In some particularly preferred embodiments, the second polysaccharide is nanocellulose. In some preferred embodiments, the nanocellulose is included in the aqueous mixture at a weight/weight percent of from 1.0% to 10.0% and most preferably from 1.0% to 3.0%.

In some preferred embodiments, the nanocellulose used to form the matrix further comprises mannitol. In some preferred embodiments, the nanocellulose is optionally supplemented with mannitol at a weight percent of from 1.0% to 10.0% and most preferably from 2.0% to 7.0%.

In some particularly preferred embodiment, the present invention provides an article comprising a solid matrix formed from a mixture of alginate and nanocellulose, said matrix further comprising a heat-treated salmonid egg extract, wherein the weight/weight percent of the alginate in the article is from 1.0% to 10.0%, most preferably from 1.0% to 3.0%, the weight/weight percent of the nanocellulose in the article is from 1.0% to 10.0%, most preferably from 1.0% to 3.0%, and the volume/weight percent of the heat-treated Salmo salar egg extract in the article is from 5.0% to 20.0%, most preferably from 8.0% to 16.0%.

In some preferred embodiments, one or more additional active agents may be included in the matrix. In some preferred embodiments, the one or more additional active agents are incorporated into the aqueous mixture prior to formation of the matrix.

Suitable additional active agents include, but are not limited to, non-steroidal anti-inflammatory drugs (NSAIDS) (the NAIDS can, for example, be selected from the following categories: (e.g., propionic acid derivatives, acetic acid derivatives, fenamic acid derivatives, biphenylcarboxylic acid derivatives and oxicams)); steroidal anti-inflammatory drugs including hydrocortisone and the like; antihistaminic drugs (e.g., chlorpheniranune, triprolidine); antitussive drugs (e.g., dextromethorphan, codeine, carmiphen and carbetapentane); antipruritic drugs (e.g., methidilizine and trimeprizine); anticholinergic drugs (e.g., scopolamine, atropine, homatropine, levodopa); anti-emetic and antinauseant drugs (e.g., cyclizine, meclizine, chlorpromazine, buclizine); anorexic drugs (e.g., benzphetamine, phentermine, chlorphentermine, fenflurarnine); central stimulant drugs (e.g., amphetamine, methamphetamine, dextroamphetamine and methylphenidate); minoxidil; antiarrhythmic drugs (e.g., propanolol, procainamide, disopyraminde, quinidine, encainide); P-adrenergic blocker drugs (e.g., metoprolol, acebutolol, betaxolol, labetalol and timolol); cardiotonic drugs (e.g., milrinone, amrinone and dobutamine); antihypertensive drugs (e.g., enalapril, clonidine, hydralazine, minoxidil, guanadrel, guanethidine); diuretic drugs (e.g., amiloride and hydrochlorothiazide); vasodilator drugs (e.g., diltazem, amiodarone, isosuprine, nylidrin, tolazoline and verapamil); vasoconstrictor drugs (e.g., dihydroergotamine, ergotamine and methylsergide); antiulcer drugs (e.g., ranitidine and cimetidine); anesthetic drugs (e.g., lidocaine, bupivacaine, chlorprocaine, dibucaine); antidepressant drugs (e.g., imipramine, desipramine, amitryptiline, nortryptiline); PDE5 inhibitors such as Viagra® or Cialis®; tranquilizer and sedative drugs (e.g., chlordiazepoxide, benacytyzine, benzquinamide, flurazapam, hydroxyzine, loxapine and promazine); antipsychotic drugs (e.g., chlorprothixene, fluphenazine, haloperidol, molindone, thioridazine and trifluoperazine); antimicrobial drugs (antibacterial, antifungal, antiprotozoal and antiviral drugs).

Antimicrobial drugs which are preferred for incorporation into the present composition include, for example, pharmaceutically acceptable salts of β-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, hexamidine isothionate, metronidazole; pentamidine, gentamycin, kanamycin, lineomycin, methacycline, methenamine, minocycline, neomycin, netilmycin, paromomycin, streptomycin, tobramycin, miconazole, and amanfadine.

Other drug moieties of use in practicing the present invention include antineoplastic drugs (e.g., antiandrogens (e.g., leuprolide or flutamide), cytocidal agents (e.g., adriamycin, doxorubicin, taxol, cyclophosphamide, busulfan, cisplatin, a-2-interferon) anti-estrogens (e.g., tamoxifen), antimetabolites (e.g., fluorouracil, methotrexate, mercaptopurine, thioguanine).

The compositions can also comprise hormones (e.g., medroxyprogesterone, estradiol, leuprolide, megestrol, octreotide or somatostatin); muscle relaxant drugs (e.g., cinnamedrine, cyclobenzaprine, flavoxate, orphenadrine, papaverine, mebeverine, idaverine, ritodrine, dephenoxylate, dantrolene and azumolen); antispasmodic drugs; bone-active drugs (e.g., diphosphonate and phosphonoalkylphosphinate drug compounds); endocrine modulating drugs (e.g., contraceptives (e.g., ethinodiol, ethinyl estradiol, norethindrone, mestranol, desogestrel, medroxyprogesterone), modulators of diabetes (e.g., glyburide or chlorpropamide), anabolics, such as testolactone or stanozolol, androgens (e.g., methyltestosterone, testosterone or fluoxymesterone), antidiuretics (e.g., desmopressin) and calcitonins).

Also of use in the present invention are estrogens (e.g., diethylstilbesterol), glucocorticoids (e.g., triamcinolone, betamethasone, etc.) and progenstogens, such as norethindrone, ethynodiol, norethindrone, levonorgestrel; thyroid agents (e.g., liothyronine or levothyroxine) or anti-thyroid agents (e.g., methimazole); antihyperprolactinemic drugs (e.g., cabergoline); hormone suppressors (e.g., danazol or goserelin), oxytocics (e.g., methylergonovine or oxytocin) and prostaglandins, such as mioprostol, alprostadil or dinoprostone, can also be employed.

Other useful active compounds include immunomodulating drugs (e.g., antihistamines, mast cell stabilizers, such as lodoxamide and/or cromolyn, steroids (e.g., triamcinolone, beclomethazone, cortisone, dexamethasone, prednisolone, methylprednisolone, beclomethasone, or clobetasol), histamine H₂ antagonists (e.g., famotidine, cimetidine, ranitidine), immunosuppressants (e.g., azathioprine, cyclosporin), etc. Groups with anti-inflammatory activity, such as sulindac, etodolac, ketoprofen and ketorolac, are also of use. Other drugs of use in conjunction with the present invention will be apparent to those of skill in the art.

In some preferred embodiments, the articles of the instant invention find use in the treatment of wounds. Accordingly, in some preferred embodiments, the articles of the instant invention may preferably be topically applied to a wound of a subject. The present invention is not limited to the treatment of any particular type of wound. In some preferred embodiments, the wound is a burn wound. Types of burn wounds that may be treated include partial thickness burn wounds (second degree burns), superficial burn wounds (first degree burns) and full thickness burn wounds (third degree burns). In some particularly preferred embodiments, the burn wound is a partial thickness burn wound. In other embodiments, the wound is a chronic wound. In particular, the chronic wounds may be associated with diabetes (i.e., diabetic ulcers such as diabetic foot ulcers), obesity, spinal cord injury (pressure ulcers), venous ulcers, and the like. As such, in some preferred embodiments, the chronic wound is a diabetic ulcer, venous ulcer, pressure ulcer, or ischemic ulcer. In still other preferred embodiments, the wound may be an abrasion, a skin tear, a puncture wound, a surgical wound or incision, or a laceration. In other preferred embodiments, the wound may be an insect bite or sting.

### EXAMPLES

### Example 1

### Preparation of Fish Egg Extracts

Fresh, unfertilized salmon (Salmo salar) eggs harvested from females in reproductive phase (late fall) are kept on ice, and the extract preferably made immediately. The eggs are crushed and eggshells is removed by a sieve. 50% v/v 0.9% NaCl is added to the filtrate. The diluted filtrate is heated to 90 degrees Celsius for 20 minutes with stirring every 5 minutes. The mixture is then centrifuged at 4500 rpm for 10 minutes. The supernatant is then transferred to a Stericup Quick Release vacuum driven disposable filtration system (0.22 mM) and a vacuum applied. The resulting filtrate (HTX) is aliquoted and frozen for use. The HTX has a protein content of from 100 to 110 mg/ml.

### Example 2

### Use of 3-d Matrix Containing HTX (Collex Matrix) to Treat Burn Wounds

### Methods

Production of Collex and Collex without HTX. The ink for production of the Collex matrix contains Alginate (2% (w/w), MVG, NovaMatrix AS), Nanocellulose supplemented with 4.6% Mannitol (1.7% (w/w), OceanTunicell AS), HTX (12% (v/w), Regenics AS), NaCl (0.9% (w/v) Fresenius Kabi), CaCl₂) (0.02M, SigmaAldrich). Approximately 200 μl of this ink (ca. 200 mg) was 3D printed using a 4th Gen 3D Bioplotter (EnvisionTEC, Gladbeck, Germany) into 4 layers measuring 16×16 mm. The patch was crosslinked with 0.02M CaCl₂) solution supplemented with saline (0.9% NaCl) and HTX (12%). The Collex matrix was kept in a polyethylene/aluminum sachet with an argon atmosphere and stored in a fridge prior to analysis. Production of the Collex matrix without HTX was identical to the Collex matrix, except HTX was replaced by saline (0.9% NaCl, Fresenius Kabi).

Endotoxin assay. The Endotoxin levels were tested in five Collex matrix dressings of sizes between 190 and 197 mg. The Collex matrix was extracted in 0.9% NaCl in a total volume of 2 ml (1:10 dilution), incubated for 1 hour at 37° C., at 50 rpm. The tubes (containing dressing and extract) were then centrifuged for 30 minutes at 4000 rpm. 1 ml of the supernatant/extract at 1:10 ratio was transferred into the new tube. Subsequently, the supernatant/extract was further diluted 1:100 (0.1 ml of extract+0.9 ml of water (Biowest)), and the endotoxin levels tested by PyroGene™ kit (Lonza, #50-658U) and the WinKQCL™ Endotoxin Detection & Analysis Software. The analysis was completed according to the European Pharmacopoeia 2.6.32: Test for bacterial endotoxins using recombinant factor C.

Animals: Housing, anesthesia, and pain treatment. The study was performed in three female Göttingen minipigs from Ellegaard Göttingen Minipigs A/S. The housing of the animals was in accordance with EU Directive 2010/63/EU of 22 Sep. 2010 on the protection of animals used for scientific purposes. On the day of wounding, anesthesia was achieved by an intramuscular injection in the neck (1.0 mL/10 kg) of a mixture of Zoletil 50®Vet., Virbac, France (125 mg tiletamine and 125 mg zolazepam), 20 mg xylazine/mL (6.25 mL), 100 mg ketamine/mL (1.25 mL) and 10 mg butorphanol/mL (2.5 mL). After induction of anesthesia and preparation of the animal for surgery the animal was intubated, and the anesthesia maintained by isoflurane.

For treatment of post-surgical pain, the animals were given a transdermal dressing with Fentanyl (75 μg/hour) for up to 72 hours from the day before surgery. In relation to the wounding procedure, the animals were given an intramuscular injection of methadone (10 mg/mL, 0.02 mL/kg). In addition, the animals were given an intramuscular injection of 20 mg meloxicam/mL, (0.02 mL/kg) on the day of wounding (Day 1). For the following two days, treatment with 15 mg meloxicam/mL oral suspension continued once daily by oral administration. During the first week following wounding and in relation to collection of biopsies on Day 10, the animals received an intramuscular injection of buprenorphine (0.3 mg/mL, 0.04 mL/kg) prior to collection of biopsies, dosing and change of bandage.

The dosing and dressing changes were performed under general anesthesia. Prior to collection of biopsies, dosing and dressing change, the animals were anaesthetized with propofol given intravenously (via a perfusion line) in an ear vein catheter until effect (absence of corneal reflexes and good muscle relaxation, typically 2-2.5 mg/kg, 10 mg/mL, i.e., 0.2-0.25 mL/kg). Before use of propofol, the TauroLock™ Hep500 was withdrawn. On the day of necropsy (Day 23), the animals were weighed, examined externally and anaesthetized by an intramuscular injection in the neck or in the left hind leg (about 0.3 mL per kg body weight) of a mixture of Zoletil 50 Vet., Virbac, France (125 mg tiletamine and 125 mg zolazepam), 20 mg xylazine/mL (6.25 mL), 100 mg ketamine/mL (1.25 mL) and 10 mg butorphanol/mL (2.5 mL). The animals were terminated by exsanguination and were not fasted prior to necropsy.

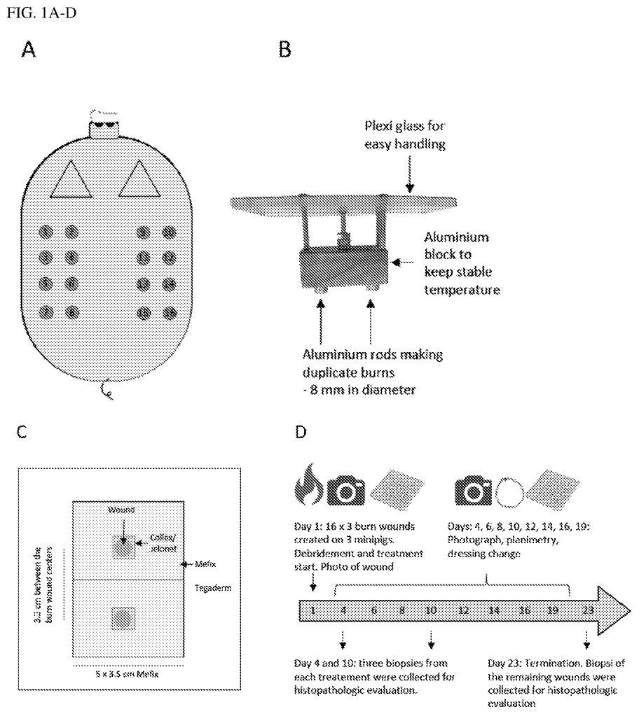

Burn procedure. The wounding procedure was performed while the animals were under anesthesia. The dorso-lateral area of both sides of the back of the animal were clipped and further shaved using an electric shaver/trimmer, washed with soap and water, and rinsed with gaze soaked in sterile water. The area was disinfected with 70% ethanol and 5% iodine ethanol. On Day 1, sixteen circular partial thickness burn wounds (8 mm in diameter) were established on the back of each animal, eight on each side of the spine (FIG. 1A). The burns were created with a custom-made aluminum block consisting of two cylinders (diameter 8 mm, FIG. 1B). The aluminum block was attached by screws to an acrylic plate for easier handling, with the assembly weighing approximately 111 g. The aluminum block was initially preheated in boiling water. The heated aluminum block was wiped dry. Immediately after, the heated aluminum block was placed on the animal with the two cylinders directly in contact with the skin surface for a period of 25±5 seconds.

Dressings and dressing changes. Production of the Collex matrix and Collex matrix without HTX is described above. Jelonet® vaseline compress dressing was cut in squares with the size matching the Collex matrix and Collex matrix without HTX (1.6×1.6 cm). All three dressings are non-adherent and were fixed by Mefix from Mölnlycke (5×3.5 cm) and Tegaderm from 3M (10×10 cm) as outlined in FIG. 1C. On top of these supportive dressings, Fixumull from BSN was used and a netlike body stocking from BSN attached to a neck collar.

The dressings were changed at Day 4, 6, 8, 10, 12, 14, 16, 19 and 23. At each dressing change, the wounds were evaluated macroscopically, by planimetric drawings, and by photos. At Day 4 and Day 10, three wounds from each treatment were biopsied and terminated. At the end of the experiments, all wounds were biopsied and sent for histopathologic evaluation. The timeline is illustrated in FIG. 1D.

Processing and microscopic examination. On Day 4 and 10, collection of biopsies took place while the animals were under anesthesia due to dressing change (FIG. 1D). The biopsies were collected by an 8 mm punch biopsy and included tissue from the center and edge of the wound as well as part of unaffected skin surrounding the wound. At necropsy, all wounds were cut free as a block (approximately 1 cm margin around the wound and if possible 0.5 cm margin to the bottom) separated from skeletal muscle tissue. All biopsies were fixed in phosphate buffered neutral 4% formaldehyde. After fixation, representative specimens from the tissues specified for microscopic examination was trimmed and processed. The specimens were embedded in paraffin and cut at a nominal thickness of approximately 5 μm. The slides were stained with haematoxylin and eosin and examined by a Scantox A/S study pathologist under a light microscope. Slides were evaluated in accordance with ISO 10993-6:2016. The scoring system was semi-quantitative, and microscopic evaluation was performed using a light microscope with an eyepiece-mounted grid to aid in the count of features of the histological sections. The depth of the burn wound was evaluated based upon damage to the epidermis and dermis and damage to the pilo-sebaceous appendages. Reactivity scores were based on degree of several factors: Polymorphonuclear cells, lymphocytes, macrophages, giant cells, necrosis, neovascularization, fatty infiltrate, edema, hemorrhage, mineralization, dilated blood vessels, crust, and hyperkeratosis.

Planimetric evaluation and photographs. The outlines of the wound edge and areas covered with slough/eschar, hard crusts, granulation tissue or epithelium were drawn on transparent sheets at listed days (FIG. 1D). The sheets were scanned, and an algorithm was developed to automatically measure the area of the wound. The photographs were taken with digital camera in a standardized manner, with flash, at a fixed distance. Square metal boxes were used to calibrate the distance.

Cultivation and differentiation of monocyte to macrophage cells. THP-1 monocyte cell line (ATCC, TIB-202) were cultured in RPMI (Lonza, 12-702F) supplemented with 10% fetal bovine serum (ATCC, 30-2025) and 1% Penicillin-Streptomycin (Sigma Aldrich, P4333). This is from now on called “medium”. Cells were kept at 37° C., 5% CO2, humidified atmosphere. For differentiation of THP-1 into M0 and M1, the protocol used was essentially as described in 22: Cells were seeded at a concentration of 2×10⁵ cells/ml in 24 well plates and exposed to Phorbol 12-muristate 13-acetate (PMA, 200 nM, Sigma-Aldrich, P8139) for 24 hours. This transformed the cells from monocyte in suspension to adherent macrophages (M0). Medium was removed from M0 cells and replaced by medium containing interferon (IF)-g (20 ng/ml, Sigma-Aldrich, GF305) and Lipopolysaccharide (LPS, 250 ng/ml, Sigma-Aldrich, P4391) and incubated for 24 hours causing an M0 to M1 differentiation. Collex matrix (ca 200 mg) and Collex matrix without HTX (ca 200 mg) was soaked in medium (1 ml) and incubated at 37° C. for 4 hours. This causes a release of the HTX content from Collex matrix into the medium at a protein concentration similar to 2% HTX. In parallel, HTX (2%) and IL-4 (40 ng/ml) was prepared. Medium containing IFNγ and LPS was replaced by medium containing Collex matrix release, Collex matrix without HTX release, HTX, IL-4 or medium only and incubated for an additional 48 hours. Supernatant from these cells were tested for levels of interleukin (IL)-1B, and the cells were tested for cell death and oxidative stress.

Interleukin-1 β ELISA assay. The level of IL-1β was determined from the medium of the cell cultures by Human IL-1 beta ELISA Kit (Abcam, ab214025) according to the manufacturer's recommendations.

Propidium iodide (PI) staining. PI (Sigma Aldrich, P4170), a fluorescent DNA-binding dye, was used to determine cell death. It acts by penetrating cell membranes of dead and dying cells. 48 hours after HTX, Collex matrix or Collex matrix without HTX treatment, cells were detached by scraping and pipetting to obtain single cells and transferred to Eppendorf tubes on ice. PI was added to the tubes (final concentration 1 mg/ml), and the signal analyzed immediately by flow cytometry (MACSQuant Analyzer 10 Flow Cytometer; Miltenyi Biotec) by excitation at 2=488 nm and detection at A=585/40 nm.

Reactive oxygen species (ROS) staining. CellROX green (Thermo Fisher, C10492) was used for detection of ROS, and is a dye converted to fluorescent molecules upon intracellular oxidation. 48 hours after HTX, Collex matrix or Collex matrix without HTX treatment, cells were detached by scraping and pipetting to obtain single cells and transferred to Eppendorf tubes. CellROX green was added to the tubes (final concentration 5 M), and the tubes incubated at 37° C. for 30 min. The signal intensity was analyzed by flow cytometry (MACSQuant Analyzer 10 Flow Cytometer; Miltenyi Biotec) by excitation at)=488 nm and detection at λ=525/50 nm.

Statistical analysis. All statistical analysis was performed using GraphPad Prism software, version 9.4 (GraphPad Software, La Jolla, CA). The significance between the treatment groups were determined by unpaired T-test and statistical significance was defined as P value <0.05. In the animal experiment, planimetric evaluation from Day 6 is excluded from the analysis because the planimetric drawings were performed by a different veterinarian and showed a markedly reduced wound area of all wounds compared to the other days. In addition, two outlier wounds were identified (one from Collex matrix and one from Jelonet® treatment) and removed from the analysis in agreement with Scantox A/S. One outlier appeared substantially more burned than the other 47 wounds, and one outlier was subjected to erroneous drawing of the wound border at planimetric evaluation.

### Results

Collex is sterile and shows biocompatibility when used on burn wounds in minipigs. The Collex matrix, tested in this report, is an all-marine 3D printed dressing in which alginate and nanocellulose are combined with the salmon roe extract, HTX. When Collex matrix is added to any solution, HTX is released from the dressing to the surrounding solution (data not shown). We have previously shown that HTX accelerates healing of partial thickness burn wounds in a human explanted skin model,23 and have here combined the wound healing qualities of HTX with the wound healing qualities of alginate and nanocellulose.

HTX contains a high degree of proteins and lipids and is sensitive to oxidation and excessive heat. Therefore, conventional sterilization of medical devices such as the exposure to Ethylene oxide and autoclavation could not be used for Collex matrix (24). Instead, sterile ingredients were used, and the production was finalized as sterile as possible. Before testing Collex matrix on animals, the sterility of the end products was tested by detection of endotoxins. Endotoxins are lipopolysaccharides (LPS) from the outer membrane of bacteria, and gives an estimate not only for the sterility of the product at the time of testing, but also for whether there has been bacterial contamination present during the production chain. (25) As shown in table 1, the analysis showed endotoxin levels below 0.005 EU/ml for all five Collex matrix samples analyzed, well below the limits for medical devices (0.5 EU/ml according to the Center for Devices and Radiological Health and European Pharmacopeia), strongly indicating that Collex is sterile.

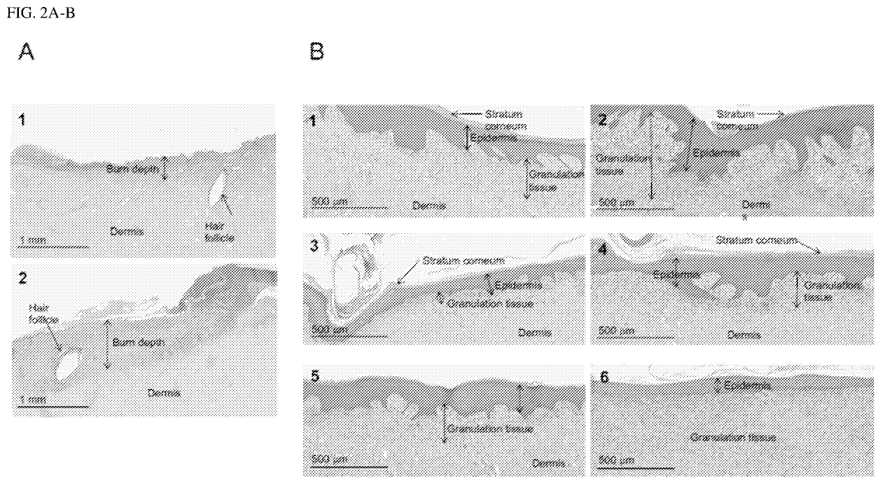

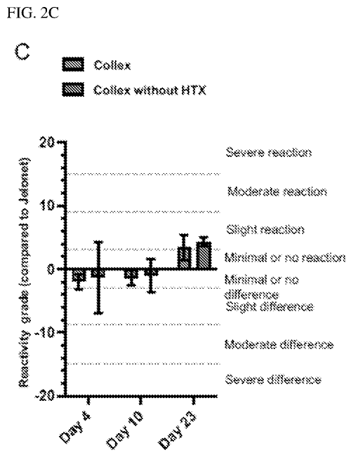

To assess the safety and efficacy of Collex matrix preclinically, it was tested on partial thickness thermal burn wounds generated on the back of Göttingen minipigs. The depth of the burn wound was determined from microscopic evaluation of haematoxylin and eosin-stained slides of biopsies from the wounds on Day 4 and illustrated by two independent wounds in FIG. 2A. The burn depth, visualized by deeper eosinophilic staining of denatured dermal collagen, extends to the hair follicles confirming the wounds inflicted were partial thickness wounds.

Collex matrix was compared to a traditional non-adherent dressing, Jelonet®, and to an alginate and nanocellulose dressing similar to Collex but with 0.9% NaCl replacing HTX. Collex matrix without HTX has the beneficial qualities of alginate and nanocellulose hydrogels without the effect of HTX, isolating any potential influence of HTX on the healing process. Collex matrix is developed according to guidelines for medical devices, and in that respect, the local effect on skin reactivity was analyzed from the haematoxylin and eosin-stained biopsy slides (according to ISO 10993-6:2016). FIG. 2B shows two representative wounds from each treatment at Day 23. Pathologist examination at Scantox A/S found that all wounds, except one wound treated with Jelonet®, were fully re-epithelized at Day 23. The photos illustrate complete epidermis on top of granulation tissue and low reactivity. Jelonet® is currently used as initial treatment for burn wounds in Norwegian hospitals, (10) and was used as a reference for reactivity. Based on the semi-quantitative scoring system, Collex matrix showed minimal or no reactivity compared to Jelonetv (FIG. 2C), indicating that it is well tolerated by the breached skin. Collex matrix without HTX showed a slight reaction on Day 23, however, this dressing is not developed for use in the clinic or in clinical testing. Furthermore, according to macroscopic assessment by veterinarians at Scantox A/S, there were no apparent infection in any of the wounds, supporting the sterility indicated by the endotoxin assay, and demonstrating the ability of Collex to protect the wound from surrounding microorganisms.

Collex matrix accelerates healing of partial thickness burn wounds in minipigs. During the healing process, each wound was evaluated macroscopically, by planimetric drawings, and by photographs. Macroscopic evaluation by Scantox A/S showed formation of hard crust on wounds treated with Jelonet® on Days 10, 12 and 14, but not on the wounds treated with Collex or Collex without HTX (data not shown), indicating that the alginate and nanocellulose dressings successfully kept the wounds moist, thereby preventing formation of scabs.

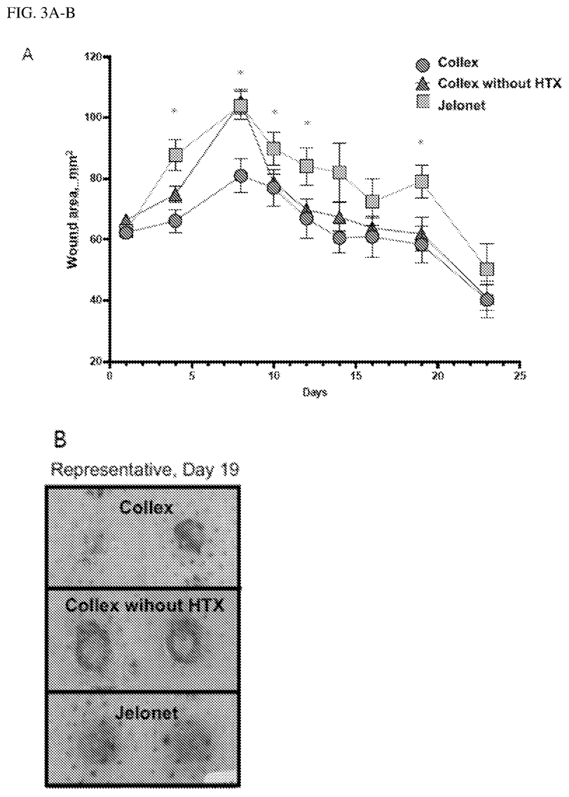

The wound area at each time point was calculated from the planimetric drawings. FIG. 3A shows average wound area of all wounds on Days 1 to 23 (end of experiment). As expected for partial thickness burn wounds, the average wound size initially increased for the first week before it gradually decreased in size. At all timepoints tested, wounds treated with Collex matrix had lower average area compared to wounds treated with Jelonet® (in particular) and Collex matrix without HTX. The difference in wound area between Collex matrix and Jelonet® was significant (grey star in FIG. 3A, p<0.05) on Days 4, 10, 12, and 19, and close to significant on Days 8 and 14 (p=0.058 and 0.055 respectively). Area of wounds treated with Collex matrix was lower than area of wounds treated with Collex matrix without HTX at all time points except Day 23 where histopathological evaluation indicated that wounds were fully healed. However, statistically significant difference was only observed on Day 8 (green star in FIG. 3A, p<0.05), and at Day 4, the difference was close to significant (p=0.078). The reduced area of wounds treated with Collex matrix indicates an accelerated wound healing.

For visual evaluation of the wounds, digital photos were captured in a standardized manner. FIG. 3B shows two representative wounds from each treatment at Day 19 when the wounds were close to being fully healed. The wounds were chosen based on the planimetric evaluation and had the areas closest to the mean area in the respective treatment category. The photos visualize a reduced wound area and reduced rubor of wounds treated with Collex matrix compared to both wounds treated with Collex matrix without HTX and wounds treated with Jelonet®, supporting an accelerated wound healing as indicated by the planimetric evaluation.

Collex matrix reduces inflammation in partial thickness burn wounds in minipigs. In partial thickness and full thickness burn wounds, wounds may progress even after the thermal exposition due to secondary necrosis caused by excessive inflammation. (4) From Day 1 to Day 4, wounds treated with Collex matrix only increased in size by 8%, whereas wounds treated with Collex matrix without HTX and Jelonet® increased by 14% and 40% respectively (FIG. 3A). Further, from Day 1 to 8, wounds treated with Collex matrix increased in size by 42%, whereas wounds treated with Collex matrix without HTX and Jelonet® increased in size by 59% and 66% respectively. Moreover, the difference in size between wounds treated with Collex matrix versus Jelonet® and Collex matrix without HTX was most pronounced in the first week. At Day 4, treatment with Collex matrix caused a reduction in wound size of 23% compared to wounds treated with Jelonet®, and 11% compared to Collex matrix without HTX. At Day 8, treatment with Collex matrix caused a reduction in size of 15% and 16% compared to wounds treated with Jelonet® and Collex without HTX respectively.

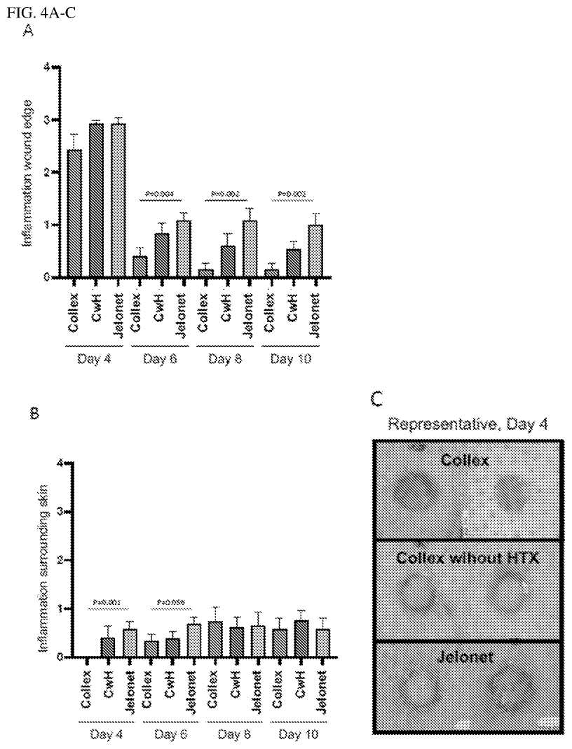

During each dressing change, the wounds were evaluated macroscopically by veterinarians at Scantox A/S. In this evaluation, inflammation was assessed for each wound and given a score from 0 (not present) to 4 (marked). FIG. 4A shows the average scores for inflammation at the wound edge. The inflammatory response was evaluated to be 17% reduced on Day 4 for wounds treated with Collex matrix, compared both to wounds treated with Collex matrix without HTX and with Jelonet®. On Day 6, overall inflammation was reduced, but was markedly more reduced in wounds treated with Collex matrix compared to wounds treated with Collex matrix without HTX and Jelonet® with 51% and 62% reduction respectively. On Day 8 and Day 10, the level of inflammation was essentially similar to Day 6, with wounds treated with Collex matrix displaying the lowest levels of inflammation. Inflammation in the surrounding skin was low (FIG. 4B), however, at Day 4 and Day 6, significant reduction of inflammation was seen around wounds treated with Collex matrix compared to wounds treated with Jelonet®. On Day 8 and Day 10, there were low levels of inflammation in the surrounding skin of all wounds, and no differences between the treatments were observed.

In addition to the macroscopic evaluation, histopathologic assessment of hematoxylin and eosin-stained biopsy slides of wounds from Day 4 and Day 10 (timeline is shown in FIG. 1D) was performed. This analysis showed a slightly reduced inflammatory response in the wounds treated with Collex matrix compared to wounds treated with Collex matrix without HTX and Jelonet® (data not shown). With a small sample size (N=3) and large variations, statistically significant values were not obtained. However, the slight indication of reduced inflammation agrees with the reduced inflammation observed by the macroscopic evaluation. The excessive inflammation at Day 4 can also be visualized by the red wound border in the photos. FIG. 4C shows two representative wounds in each treatment category at Day 4 (the same wounds as shown in FIG. 3B). The photos illustrate the reduced inflammatory response in wounds treated Collex matrix compared to wounds treated with Collex matrix without HTX and Jelonet®, supporting the observations indicated from macroscopic and histopathologic assessment.

Evaluated collectively, the results from this minipig burn trial indicate that Collex matrix accelerates healing of partial thickness burn wounds, where dampening of the inflammatory response in the initial phase of wound healing is likely to be a significant contributing factor.

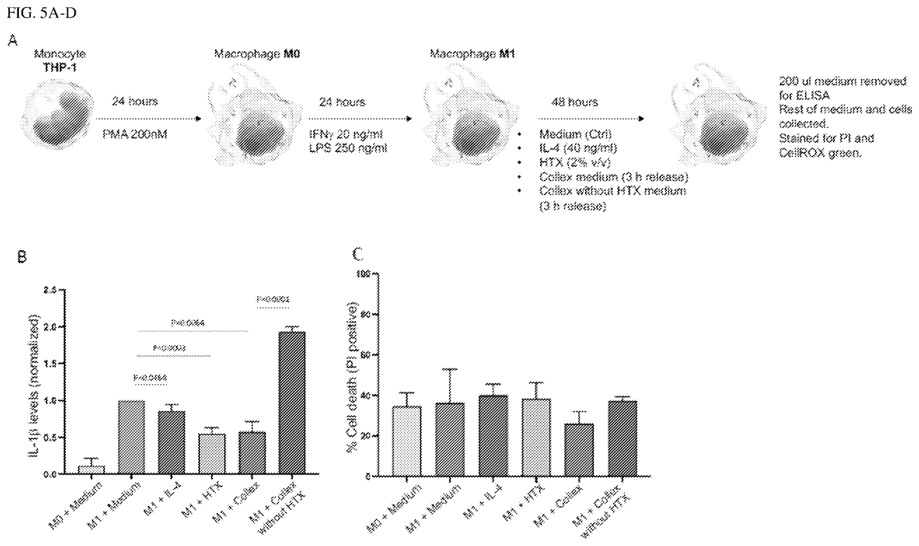

Collex matrix and HTX reduce the inflammatory response and the level of oxidative stress in activated M1 macrophages. Because results from the burn study on minipigs strongly indicates that Collex matrix accelerates wound healing by reducing inflammation, we aimed to study the anti-inflammatory response in more detail. To this end, we employed an immortalized, human monocyte cell line THP-1. THP-1 is widely used in the literature and can be easily differentiated into M0 macrophages by phorbol 12-myristate 13-acetate (PMA) and further into pro-inflammatory M1 by interferon gamma (IFNγ) and LPS.22 M1 macrophages secrete pro-inflammatory cytokines such as IL-1β 26 To determine the effect of Collex matrix as well as HTX on inflammation, we therefore stimulated THP-1 into the pro-inflammatory M1 state and treated these cells with: medium exposed to Collex matrix; medium exposed to Collex matrix without HTX; HTX; interleukin 4 (IL-4) for 48 hours (FIG. 5A). The level of secreted IL-1β in the medium was then analyzed by ELISA. HTX diluted to 2% in medium results in a similar HTX concentration as Collex matrix (0.2 g) soaked in medium (1 ml). IL-4 is reported to prime the transition of pro-inflammatory M1 macrophages to the anti-inflammatory M2 phenotype, hence reducing the level of IL-1B 27,28 and was therefore used as a reference in the assay. As shown in FIG. 5B, differentiation of cells from M0 (PMA only) to M1 (PMA+IFNγ+LPS) markedly increased the level of IL-1β as expected. Importantly, treatment with both Collex matrix and HTX markedly reduced secretion of IL-1B by 45% and 43% respectively. Collex matrix without HTX, however, did not cause reduced levels, but rather enhanced secretion of IL-1B, strongly indicating that HTX causes the anti-inflammatory response of Collex matrix. Treatment of M1 cells with IL-4 slightly reduced secretion of IL-1B (14%). The reduction was below the expected level as the IL-4 dose was at the upper end of the doses used in the referenced literature on the same cell line (22,27). However, optimization of IL-4 dosage was not within the scope of this paper. To ensure that the indicated reduced secretion of IL-1β was not merely a reduced cell survival, cells from the same experiment were collected, stained with the cell death dye propidium iodide (PI) and analyzed by flow cytometry. As shown in FIG. 5C, neither HTX nor Collex matrix caused an increase in cell death. In fact, the degree of cell death was slightly reduced for cells exposed to medium from Collex.

It is well established that burn wounds cause excessive release of reactive oxygen species (ROS), causing damage to DNA, proteins, lipids and subsequently the surrounding tissue (7). Moreover, ROS is known to be pro-inflammatory (29). Because the burn wounds treated with Collex matrix appeared to have reduced inflammation, and because HTX (12% of Collex) contains chemical substances known to have antioxidant properties, we hypothesized that Collex matrix could also reduce ROS in activated macrophages. To test this, the same M1-polarized macrophages as used in the IL-1β and cell death assay were stained with the ROS dye CellROX. As shown in FIG. 5D, both Collex and HTX significantly reduced the ROS levels by 48% and 41% respectively. Treatment with IL-4 did not cause any reduction in ROS, suggesting that the IL-1B reduction from IL-4 treatment was unrelated to the ROS levels. Furthermore, treatment with Collex matrix without HTX did not cause reduced levels of ROS, strongly indicating that the antioxidant effect of Collex was caused by HTX. In summary, the anti-inflammatory effect of Collex matrix, indicated from the animal study, is supported by in vitro findings, showing reduced levels of both IL-1β and ROS.

### DISCUSSION

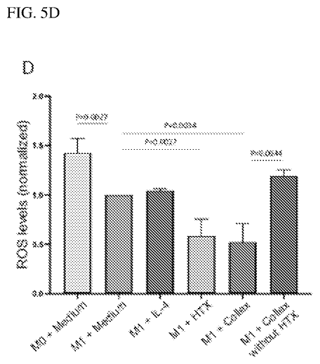

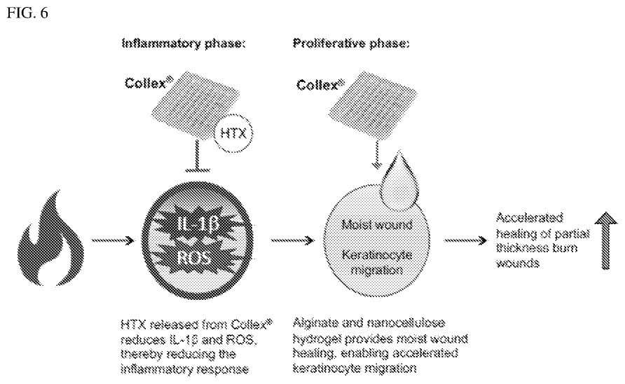

The inventors have shown that dermal treatment of partial thickness porcine burn wounds with Collex matrix causes reduced inflammation, reduced wound progression, provides a moist wound environment, prevents scab formation, and accelerates wound closure. In addition, Collex matrix appears to be biocompatible and to protect the wounds from infection. In vitro data on pro-inflammatory macrophages support the anti-inflammatory effect indicated in the minipig trial. FIG. 6 shows a model of the suggested mode of action of Collex matrix. Burn wounds cause an excessive inflammatory response with high levels of IL-1β and ROS. Both inflammation and ROS are tightly associated with increased apoptosis and necrosis, hence contributing to progression of burn wounds (4,5,30,31). HTX released from Collex matrix appears to suppress inflammation by reducing the level of IL-1β and the level of ROS. Stock HTX at a concentration of 2% (v/v) results in a similar concentration of HTX compared to Collex soaked in medium. Given that stock HTX and Collex matrix reduced the production of IL-1B and of ROS to similar levels, it is highly likely that the anti-inflammatory and antioxidant effect of Collex matrix is caused by HTX. In the proceeding proliferative phase of burn wound healing, Collex matrix provides a moist environment that is favorable for keratinocyte migration (32). It is likely that these two mechanisms of Collex matrix combined cause an accelerated wound healing of partial thickness burn wounds.

Among current dressings indicated for partial thickness burn wounds, there are some common properties that are generally regarded as important: The dressing should cover and protect the wound, the dressing should be absorbent to manage exudates, and it should be moist to accelerate re-epithelization. These are all mechanical properties important to facilitate an optimal environment for wound closure and for reducing the risk of infection during healing (1,2). Collex matrix shares all these properties, and they are crucial for the dressing's wound healing properties; it is the authors' hypothesis that collectively, they make up the primary mode of action. Evaluating the graph in FIG. 3A supports this, where both Collex matrix and Collex matrix without HTX showed reduced wound areas compared to Jelonetv, with less pronounced differences between the two. The ability of Collex matrix to keep the wound bed moist is supported by the macroscopic evaluation, where hard crusts were identified on wounds treated with Jelonet®, but not on any of the wounds treated with Collex matrix or Collex matrix without HTX. The link between moist wound healing and prevention of crust formation and enhanced rate of re-epithelialization is well established (32,33).

The anti-inflammatory and antioxidant actions shown in this study thus appear to be ancillary modes of action of Collex matrix. However, these properties remain important, because they fulfill an unmet medical need in wound management, and are not typically found in dressings in regular use for burn management today (10,34). Excessive ROS is known to activate the inflammatory response and vice versa, hence reducing ROS may by itself reduce inflammation. Both inflammation and ROS not only cause burn wound progression and impair wound healing, but are also associated with hypertrophic scarring seen after burn wounds (35,36). It is therefore not surprising that development of novel burn care therapies focus on both reduction of the inflammatory response (3,4,37) and reduction of ROS levels (5,9,14,38,39). The anti-inflammatory action of Collex matrix, which reduces secondary necrosis, appear to give wounds treated with Collex matrix about a week's head start in terms of wound closure compared to wounds treated with Jelonet®. If this could result in faster healing of severe burns, the duration of treatment might be reduced, and patient management might more rapidly be transferred to lower tiers of care, e.g. from intensive care through inpatient and to outpatient care. These benefits could potentially lead to better patient burn care and significant healthcare provider savings.

In Norwegian hospital standard of care, Jelonet® is used in initial burn wound management 10. Given the potent anti-inflammatory effect of Collex matrix, replacing Jelonet® with Collex matrix in the early post-injury phase could reduce secondary necrosis and give faster wound closure. Moreover, Collex matrix may be an efficient dressing in treatment of chronic wounds. Chronic wounds suffer from chronic inflammation and excessive ROS (15,46), not only for the first week as for partial thickness burn wounds, but for several weeks and even months (47). Collex matrix is not yet tested on chronic wounds, however, based on results from the minipig trial, the in vitro data, and the nature of chronic wounds, we have reasons to believe that topical treatment of chronic wounds with Collex matrix would reduce inflammation and accelerate the rate of healing.

TABLE 1
Endotoxin analysis: Endotoxin values were within the limits
for medical devices (0.5 EU/ml, according to the Center
for Devices and Radiological Health and European Pharmacopeia)
of all five dressings tested. PCC recovery should be between
50 and 200% for the test to be reliable.

| | Weight (g) | EU/ml | PPC rec. | Status |
|---|---|---|---|---|
| Water (Biowest) | — | <0.005 | 66% | Extraction control |
| NaCl (0.9%) | — | <0.005 | 77% | Extraction control |
| Collex HTX 1 | 0.196 | <0.005 | 86% | PASS (<0.5) |
| Collex HTX 2 | 0.197 | <0.005 | 85% | PASS (<0.5) |
| Collex HTX 3 | 0.197 | <0.005 | 85% | PASS (<0.5) |
| Collex HTX 4 | 0.190 | <0.005 | 87% | PASS (<0.5) |
| Collex HTX 5 | 0.190 | <0.005 | 90% | PASS (<0.5) |

### Example 3

### Release of HTX from Collex Matrix

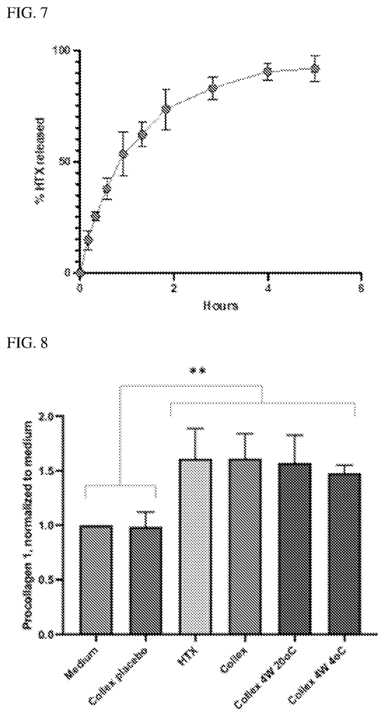

Total protein release from Collex matrix patches into DMEM supplemented with 10% FBS and 1% Pen/Strep is shown in FIG. 7. Unmodified HTX were encapsulated in the Collex patches. The total protein content was measured using absorbance at 280 nm and the absorbance from the medium (DMEM-F with Pen/Strep) was subtracted for each time point.

### Example 4

### Bioactivity of Collex Matrix Determined by Procollagen-1 Assay

The bioactivity of released HTX was tested by Procollagen I alpha 1 ELISA assay by extracting the content of Collex matrix into cell medium (DMEM-F with Pen/Strep) and exposing fibroblast cells to this cell medium for 7 days. As expected, Collex matrix without HTX (Collex placebo) did not affect procollagen-1 alpha 1 production. Collex matrix containing HTX resulted in significantly higher production of procollagen-1 alpha 1 compared to negative controls (p<0.01). A maintained bioactivity was confirmed for Collex matrix after storage for 4 weeks in refrigeration as well as room temperature, see FIG. 8.

### Example 5

### Comparison of Collex Matrix Formulations

This example describes the testing of various formulation for Collex that comprise different levels of alginate and nanocellulose.

### Materials and Methods

Ink Formulation and Optimization. The different inks were formulated by mixing medical grade TUNICELL ETC+M, enzymatically pretreated cellulose nanofibrils (E-CNF) supplemented with 4.6% mannitol (OceanTunicell AS, Bergen, Norway), H₂O, and 0.9% NaCl (Fresenius Kabi AG, Bad Homburg, Germany). Then, sodium alginate (10% w/v in saline, PRONOVA UP MVG (NovaMatrix DuPont Nutrition Norge AS, Sandvika, Norway) with Mw>200 kDa and a mannuronic/guluronic (M/G) ratio of ≥1.5, was added and after mixing left to equilibrate for 2 h before adding HTX 12% (v/v), a purified salmon roe extract (Regenics AS, Oslo, Norway), and mixed once more. All mixing steps were performed by hand for 5 min and the ink was then centrifuged to release air bubbles. The ink composition was systematically varied, as in Table 2. The ink properties were evaluated based on rheology, printability, shape fidelity, handling, and protein release profiles. To better understand and model the responses the experiments were planned according to a full factorial design (R1-R4) with R5 the center point. The data was evaluated in Statistica ver. 14.0.0.15 (StatSoft Europe GmbH, Hamburg, Germany) using the design of experiments (DOE) module.

TABLE 2
The composition of the evaluated ink variations
presented as percentage (v/v).

| Ink | Alginate (%) | Nanocellulose (%) | HTX (%) |
|---|---|---|---|
| R1 | 0.5 | 1.0 | 12 |
| R2 | 2.0 | 1.0 | 12 |
| R3 | 0.5 | 1.7 | 12 |
| R4 | 2.0 | 1.7 | 12 |
| R5 | 1.0 | 1.4 | 12 |

Ink Rheology. The characterization of the flow behavior and viscoelastic properties of the different inks were conducted using DHR-30 rheometer (TA instruments, New Castle, DE, USA) using a 20 mm upper plate together with a lower Peltier Plate. All rheological characterization were made at 20° C. and the gap used was 500 μm for all experiments. The software TRIOS version #5.5.0.323 (TA instruments, New Castle, DE, USA) was used to process the data, and for each test, 10 points per decade were taken. A 30 s soak time was used to let the sample relax on the plate before the tests were conducted. Three replicates were made of the ink compositions and the reported data was mean±standard deviation in the graphs.

The flow behavior with respect to shear was determined with a flow sweep between 100 s⁻¹ and 0.1 s⁻¹ using steady state sensing with maximum of 10 s equilibration time and 10 s sample period using a 5% tolerance to be consecutive within 3 measurements. The stress from the flow sweep was fitted to the Herschel-Bulkley model using the software TRIOS version #5.5.0.323 (TA instruments, New Castle, DE, USA). The Herschel-Bulkley model parameter fit is shown as mean±standard error (SE). The viscoelastic properties with regards to frequency were characterized using an oscillatory frequency sweep between 0.1 and 30 Hz, within the linear viscoelastic region.

Three-Dimensional Printing of Hydrogels. Inks were prepared by mixing alginate, nanocellulose, saline, and HTX by the ratios given in Table 1. Ink patches, aiming for 200 mg of ink, were 3D printed using a 4th Gen 3D-Bioplotter (EnvisionTEC, Gladbeck, Germany) in an interlaced 90-degree continuous grid pattern composed of 4 layers measuring 16 mm×16 mm. The internal grid was defined by a lateral filament separation of 1.8 mm, and 0.7 mm from the contour using a 22-gauge sized nozzle and printing pressures and speeds ranging from 0.1-0.4 bar and 4-40 mm/s, respectively. Once the 4 layers were printed, the patch was imaged by the 3D-Bioplotter—sticking nine images together and the result used to evaluate the printability. Thereafter, ink patches were crosslinked in 0.02 M CaCl2 solution (Sigma-Aldrich, Darmstadt, Germany) supplemented with HTX (12% v/v) for at least 30 min. Meanwhile, the ink patches were kept cool and shielded from light by an aluminum foil cover until transferred to an ethanol washed polyethylene/aluminum sachet with an argon atmosphere for storage in the fridge prior to analysis. In experiments including hydrogel control patches, the HTX in both the ink and the crosslinking solution was replaced by 0.9% NaCl To estimate the patch height, the final construct was transferred and imaged once more by using the same protocol as before but after the excess of crosslinking solution had been wiped dry. The printability of each material was evaluated by flow behavior, filament size, pore size, grid uniformity, and patch height. This was performed by comparing the grid structure imaged directly after printing as well as after crosslinking occurred. Three patches of each ink type were evaluated-size estimates were performed using ImageJ 1.53t (NIH, Bethesda, MD, USA) at three random sites, respectively. The mean size (width) of the main filaments and pores were estimated while smaller pores along the contour were excluded.

Release Profile. HTX is a liquid with a complex composition rich in proteins. Proteins can easily be detected using small volumes, and release of proteins was therefore used as an indicator for the total release of HTX. The release of protein (HTX) from the crosslinked hydrogel structures was systematically studied starting with a solid disk when optimizing the ink formulation for which the protein release was analyzed using a BCA protein analysis kit. Thereafter, the release from the selected R4 ink crosslinked solid disk was evaluated in different medium of increasing complexity including: 0.9% NaCl, phosphate buffered saline (PBS), chemically simulated wound fluid (CSWF), cell medium with 10% fetal bovine serum (DMEM-F), and FBS+peptone medium (FBSpept). Finally, the protein release was evaluated for the selected R4 ink 3D printed hydrogel, Collex matrix, where the manufacturing has been described in the 3D printing section. Collex matrix (approx. 200 mg), stored in sachets for 1 to 4 weeks, was transferred individually to a 24-well plate and 1 mL of cell media (DMEM supplemented with 10% FBS and 1% Pen/Strep) was added. The antibiotics were added to match the release medium used in the bioactivity assay. The plate was incubated at 37° C., 5% CO2, humidified atmosphere. At indicated time points, 2 μL cell media was removed and the protein concentration was determined using NanoDrop™ (One/OneC, Thermo Scientific, Madison, WI, USA) at 280 nm.

Chemical Characterization. The extractions of Collex matrix were performed according to ISO 10993-12:2021 in duplicate using 0.9% NaCl in Milli-Q water and isopropanol as extraction media with an extraction ratio of 0.2 g/mL, and 5-10 patches per extraction. Patches were cleaned of excess cross-linking solution by dipping in saline for <1 s. The extractions were conducted at 50° C. for 72±2 h.

Chemical analysis with gas chromatography-mass spectrometry (GC-MS) and inductively coupled plasma mass spectrometry (ICP-MS) was performed within 24 h after the extraction was ended. The extraction procedure was followed by determination of extracted organic and inorganic components with GC-MS and ICP-MS, respectively, according to ISO 10993-18:2020.

Bioburden. Evaluation of microbial cleanliness (bioburden) of Collex matrix following the European Pharmacopeia 2.6.12: Microbial enumeration tests. The HTX solution 12% (v/v) and Collex matrix was tested for any inhibitory (antimicrobial) effect on the growth of microorganisms, see Table 3, which was included in the test and to compensate for it if needs be. All samples were handled in a laminar air flow cabinet (LAF) with a high-efficiency particulate absorbing (HEPA) air filtration system.

TABLE 3
Micro-organisms included in the evaluation.

| | American Type Culture Collection |
|---|---|
| Micro-Organisms | (ATCC) Number |
| Staphylococcus aureus | 6538 |
| Pseudomonas aeruginosa | 9027 |
| Bacillus subtilis | 6633 |
| Candida albicans | 10,231 |
| Aspergillus brasiliensis | 16,404 |

HTX and Collex were evaluated for bioburden by total aerobic microbial count (TAMC) and for total yeast and mold count (TYMC). Both products were tested in triplicate. Sterility of the environment was evaluated in parallel by negative controls of tryptone soy broth (TSB), TSB-100, and 0.9% saline spread on TSB, tryptic soy agar (TSA) and sabouraud dextrose agar, (SDA), respectively.

### Bioactivity and Biocompatibility.

Cytotoxicity. The cytotoxic potential of Collex matrix was analyzed by extraction of the test item according to ISO 10993-12:2021 and cytotoxicity testing according to ISO 10993-5:2009 Annex C (MTT cytotoxicity test) in a GLP approved test facility.

Cell Cultivation. Human fibroblast cell line, Hs 707 from ATCC (CRL-7449), was cultivated at 37° C. in a humidified atmosphere supplemented with 5% CO2. The cells were grown in Dulbeccos Modified Eagles Medium (Sigma-Aldrich, D0822) supplemented with 10% FBS (ATCC, 30-2025) and 1% Penicillin-Streptomycin (Sigma-Aldrich, P4333). For the pro-collagen I alpha 1 assay, cells at passage 2 to 5 were used.

Pro-collagen I Alpha 1 Assay. Hs 707 cells were diluted to a concentration of 12×10⁴ cell/mL and 1 mL was seeded onto 24 well plates and incubated in a cell incubator for 24 h. The following day (day 1) Collex (ca 200 mg) was transferred to cell media (1.2 mL) and placed in a cell incubator for 4 h for release of HTX. Absorbance at 280 nm was measured to ensure the protein concentration was the same as 1% stock solution of HTX or higher. On the cell plate, cell media (1 mL) was replaced with the following: new cell media, cell media with extract from Collex matrix, cell media with extract from printed control (without HTX), or cell media with 1% HTX (stock solution). The cells were incubated further in a cell incubator and at day 4 and 6, new cell media was prepared as for day 1, but on these days, only 300 μL of the cell media was replaced. At day 8, cell media was collected from each treatment and the level of pro-collagen I was determined with human pro-collagen I alpha 1 ELISA kit (ab210966, Abcam, Cambridge, UK) according to manufacturer's recommendations.

### Results

Ink Rheology. Extrusion-based 3D printing is dependent on the rheological behavior of the ink to allow for printability through the syringe. To achieve acceptable printability, the ink needs to have shear thinning to flow through the nozzle when subjected to low forces. Five ink formulations were characterized by viscometry to assess the printability and the flow of the ink formulations. In FIG. 10a, the flow sweep shows that the viscosity of R1 was the lowest among the tested inks, with an initial viscosity near 20 Pa·s at a shear rate of 0.1 s⁻¹. Additionally, R1 showed no distinct shear thinning ability—there was no decrease in viscosity for increasing shear rate, likely due to the low amount of both nanocellulose and alginate. This could result in difficulties to extrude the ink on the build plate in a controlled manner. The viscosities of R2, R3, and R5 at low shear were similar and could barely be distinguished from each other, see FIG. 10a. The ratio of alginate and nanocellulose differed between R3 and R5, but seemed to balance each other in terms of viscosity, whilst R2, with a lower nanocellulose content, resulted in slightly reduced initial viscosity despite the increased alginate content, see Table 2. The R4 ink formulation contained the highest amount of both alginate and nanocellulose, which resulted in an increased viscosity at low shear as well as increased shear thinning behavior in comparison to the others, see FIG. 10a. The viscosity of the ink increases with the polymer concentration and the shear thinning is likely to be explained by disentanglements of polymer chains when subjected to a high shear rate. The combination of these characteristics is considered positive for printing in terms of extrudability.

In addition, the ink needs to have a sufficient yield stress to retain the shape of the printed structure on the build plate. A high shear thinning ability allows for an increased printing speed to be utilized whilst the yield stress is related to the ink's ability to retain its geometry on the build plate. Generally, an increased yield stress supports printing of overhang structures with minimal risk of collapse. To gain further information regarding the non-Newtonian behavior of the ink, the shear stress was fitted to a Herschel-Bulkley model, FIG. 10b and Table 4, which disclosed that the yield stress of R4 was distinctly higher compared to the other inks. R1 showed the lowest yield stress value while R2, R3, and R5 were alike. R4 showed more than a three-fold increase in yield stress compared to the other inks, which is indicative of better shape retention once printed. The factorial model showed a significant positive interaction effect between alginate and nanocellulose.

TABLE 4
Calculated yield stresses presented with
SD for the different ink compositions.

| Ink | Alginate (%) | Nanocellulose (%) | HTX (%) | Yield Stress (Pa) |
|---|---|---|---|---|
| R1 | 0.5 | 1.0 | 12 | 6.7 ± 3.1 |
| R2 | 2.0 | 1.0 | 12 | 34.7 ± 3 |
| R3 | 0.5 | 1.7 | 12 | 28.8 ± 1.9 |
| R4 | 2.0 | 1.7 | 12 | 116.2 ± 6 |
| R5 | 1.0 | 1.4 | 12 | 33.9 ± 5.3 |

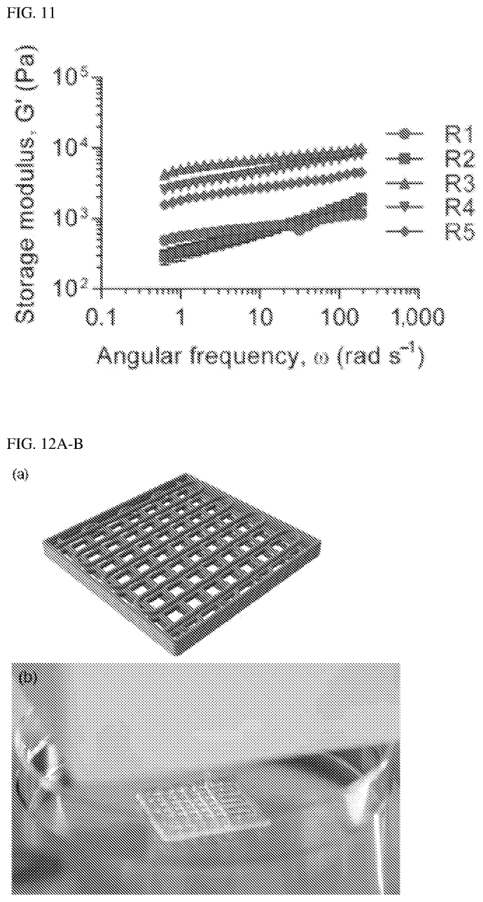

Apart from the flow behavior, which describes the rheological behavior during printing and the ability of the ink to retain its shape on the build plate, the viscoelastic property of the ink describes additional characteristics of the formulation. In this study, the storage modulus (G′), evaluated by a frequency sweep, was plotted against the angular frequency, see FIG. 11. It revealed that the ink formulations were similar with respect to angular frequency, typically related to the level of entanglement in the physical gel. The tests were conducted before crosslinking of the alginate, and no cross-over point was seen, i.e., the ink displayed a gel-like appearance, within the frequency range tested (data not shown). The main contribution to G′ was nanocellulose concentration and no other modeling of the data was performed. R1 and R2 showed a similar absolute value of G′, lower in comparison to R3-R5. The samples R3-R5 displayed a similar range in terms of G′, most likely due to their higher content of nanocellulose, see Table 2. Nanocellulose is known to have a reinforcing ability that affects the viscoelastic properties of bioinks, which has shown to be favorable for printability. The cellulose nanofibers can support the stability of the ink through their entanglements at low concentration.

Three-Dimensional Printing and Structural Characterization. The inks listed in Table 2 were evaluated for printability and shape fidelity based on the resulting overall grid uniformity, filament width, pore size, and patch height. The printing parameters of each ink were individually optimized prior to the evaluation, where patches of four layers were printed in a predetermined design and compared to the template CAD drawing, see FIG. 12 a,b.

The observations made during printing of inks R1-R5 agreed with the rheological results. A higher content of alginate, as in R2 and R4 compared to R1 and R3, respectively, resulted in improved printability and shape fidelity of the inks. This is likely due to an increased viscosity, shear thinning behavior, and yield stress. Even though R2 and R3 showed similar shear thinning ability and yield stress levels, the R3 ink displayed an improved printability and shape retention most likely due to an increased G′. The R1 and R2 tended to drip during printing and showed low to non-existing shape retention, which can be correlated to their lower yield stress and G′, which was in line with the rheological observations where R3-R5 showed increased viscoelastic properties. To achieve a continuous flow through the needle, the required printing pressure increased with the ink number going from R1 to R4 due to its increased viscosity and viscoelastic behavior. R5 required a similar pressure to R3 but needed a much higher printing speed due to the increased flow through the syringe. The HTX content was also observed to slightly improve the printability when compared to an ink where the HTX was replaced by saline, as in the control patches used in some of the other experiments such as bioactivity testing. The HTX-containing ink R4 showed better shape retention than the corresponding ink without HTX but displayed a higher tendency of air entrapment and increased hydrophobicity. This is likely to be explained by the surface activity of proteins and polar lipids in HTX, which tend to be enriched at the interphase between water and air resulting in stabilization of bubbles and increased hydrophobicity. This was evident when the patches were exposed to the crosslinking solution. The patches based on R1, R3, and R5 with a lower content of alginate appeared rather soft and sensitive, risking deformation when handled.

The patch characteristics for R1-R4, presented in Table 4, showed increasing grid uniformity, filament consistency, and conformity for increasing ink number compared to the CAD design. The structural characteristics were in line with the rheological observations, where a combination of high shear thinning, yield stress, and G′ resulted in improved 3D printing properties, as in R4, and then decreased with decreasing ink number. All the inks showed dye swell, judged by the increased filament width in comparison to the nozzle size with an internal diameter of 0.41 mm, Table 4. The printing protocol included a 20% filament overlap between layers, targeting 1.37 mm in total height. The resulting patch height estimated after crosslinking of the structure ranged between 0.5 and 1.0 mm (see Table 5), indicative of some structural collapse and flow. The patches displayed a slight shrinkage when crosslinked (not quantified). The ink could most likely accomplish a denser lattice structure and perhaps been optimized further by changing the composition by, for example, increasing the biopolymer content or by additional additives. However, given the overall printability and characteristics in combination with a maintained structural stability during storage (patches that had been stored in solution for 4 weeks were used in the bioactivity assay in 3.6), the grid structure achieved by the R4 ink was determined suitable for the given application.

TABLE 5
Characterization of the ink's printability and shape fidelity,
presented as mean values SD, n = 3 for patch height (crosslinked)
and n = 9 for filaments and pores (non-crosslinked).

| | Grid | Filament Width | Pore Size | Patch Height |
|---|---|---|---|---|
| Ink | Uniformity | (mm) | (mm) | (mm) |
| R1 | Non-existing | N/A | N/A | 0.5 |
| R2 | Low | 0.98 ± 0.19 | 0.77 ± 0.19 | 0.2 |
| R3 | Middle | 0.65 ± 0.08 | 1.19 ± 0.12 | 0.9 |
| R4 | High | 0.54 ± 0.05 | 1.24 ± 0.06 | 1.0 |
| R5 | Low | 0.83 ± 0.25 | 0.94 ± 0.25 | 0.7 |

Release Profile. The release of HTX from the crosslinked hydrogel was systematically studied starting with a solid disk design to support the optimization of the ink formulation. The protein release appeared similar regardless of the hydrogel composition within the evaluated range. Thereafter, the release was evaluated in release media of varying complexity. The loading of HTX into the hydrogels is non-specific (noncovalent), and various types of physical molecular interactions are expected to influence the binding strength of the HTX components to the gel structure. Therefore, pH, ionic strength, and protein content may affect the release rate of HTX from the hydrogels. The release profile may thus vary depending on the composition of the surrounding medium. For wound healing applications, it is important that the bioactive ingredients are released into the wound exudate, which consists of a crude mixture of proteins, salts, nutrients, and inflammatory cells and components. Saline, specifically 0.9% NaCl(aq), was utilized as a release medium in order to establish a controlled system for release. Its purpose was not to mimic the actual wound environment, but as a controlled system for initial understanding and characterization without interactions with other medium components. Additionally, phosphate buffered saline (PBS) was used as a more physiologically relevant buffer. A recipe of a chemically simulated wound fluid (CSWF) was found in the literature. The CSWF is composed of ions relevant to a wound environment as well as a protein, bovine serum albumin (BSA), in amounts that would correspond to the total protein content in a wound. It is a chemically defined medium, which offers a high level of control in in vitro assays. For the more complex protein-mixtures, fetal bovine serum (FBS) was added either in low amounts (10%) to cell culture medium with full nutritional value for cell culturing (DMEM-F), or in high amounts (50%) directly into peptone water (FBSpept); the latter is referred to as simulated wound fluid in the literature. A substantial release of HTX occurs in both protein-free PBS and in protein-rich media, such as CSWF, DMEM-F, and FBSpept. This is of importance since the hydrogel was designed to be used as a dressing applied directly on wounds, where the surrounding environment is rich in both proteins and other biological components, in which the serum has the highest resemblance with. The release results confirmed that constructs made from the R4 ink will release HTX into surrounding fluids that are similar to a real wound environment.

Finally, the release of HTX into a complex medium was studied for the selected hydrogel, Collex, 3D printed from R4 ink. Non-labelled HTX was used in the construction of 3D-printed Collex patches. Crosslinking was carried out in a CaCl₂) solution with added HTX 12% (v/v), the same concentration as in the R4 ink, to avoid HTX release losses during the crosslinking step. A time-dependent release into DMEM-F was also observed for Collex. Total protein release from Collex patches into DMEM supplemented with 10% FBS and 1% Pen/Strep to match the medium used in the bioactivity assay is shown in FIG. 7. The total protein content was measured using absorbance at 280 nm and the absorbance from the medium (DMEM-F with Pen/Strep) was subtracted for each time point. Compared to the solid disks, protein release from the 3D printed Collex patches was both faster and resulted in markedly higher total released amounts. Approximately 90% of the protein was released from the 3D-printed patch that had been crosslinked in CaCl₂) solution containing HTX. It is possible that even higher levels can be released in a wound where protein exchange occurs, which shifts the equilibrium. There were several differences between the solid disk and printed patch experiments that may explain the difference in their release profiles. With printing, there is a more precise control of the patch size, and the patches were weighed after preparation to ensure consistency between samples. In addition, they were stored for 1 to 4 weeks in sachets with storage solution prior to use. The crosslinking solution as well as storage solutions contained added HTX to avoid release from the patch. However, one cannot exclude the possibility that the patch will absorb HTX from the surrounding environment during preparation and storage, resulting in a higher total amount of HTX in the samples than expected. If that is the case, the percentage of total protein that has been released calculated based on the amount of HTX in the ink prior to printing would overestimate the actual percentage of released protein. This would be interesting to investigate further, although it would be difficult to delineate any possible absorption during storage accurately. However, such assessment is not depicting the usefulness of this patch in wound care, as long as the storage conditions are well-defined, and the patch has the desired effect when used. An important factor influencing the release rate and total released amount is the structures and surface areas of the patches; the lattice structure has a larger surface area compared to the solid disks, which makes the distance the proteins must travel through the gel structure shorter before reaching the surrounding medium. It is difficult to obtain a detailed insight in the release mechanism by only measuring protein release by absorbance at 280 nm. However, it was important to study the behavior of non-labelled HTX to allow native interactions of HTX with the gel structure, as well as using a complex medium representing a wound environment, which in combination, constrained the choice of detection methods. The results showing an effective release are encouraging.

Chemical Characterization. The chemical components of Collex were characterized to assess its safety profile, limited to volatile and semi-volatile organic compounds as well as inorganic elements. The extract solutions from Collex, either saline or isopropanol, were transparent and uncolored with no visible particles. The hydrogel remained visibly unchanged after the extraction. The extracts were analyzed in triplicate. Leached organic compounds were screened by the GC-MS method, where mainly fatty acids, cholesterol, and saccharides (predominantly mannitol) were identified in the isopropanol extracts. Measured concentrations are considered non-toxic. Leached inorganic elements identified with ICP-MS in the saline extracts of Collex patches are presented in Table 6. The reason for using two different extract solutions, saline and isopropanol, was that inorganic elements have a much higher solubility in saline compared to isopropanol, while the opposite applies for organic compounds. This was why no organic compounds and no inorganic elements were identified in the saline and the isopropanol extracts, respectively. The toxicological risk assessment following the chemical characterization revealed margins of safety that did not indicate any risk for acute systemic toxicity from using the Collex matrix. Moreover, the assessment revealed no leaching that is known to cause material-mediated pyrogenicity and concluded that leaching of compounds under clinical conditions is unlikely to cause skin irritation or sensitization.

TABLE 6
The main elements and their amount identified with ICP-MS
in saline extracts of the R4 ink hydrogel. The extraction
was performed on duplicates (a, b), and the extracts were
analyzed in triplicate presented as mean values SD, n = 3

| | Amount {ng/g Test | |
|---|---|---|
| Element | Item (a)} | Amount {ng/g Test Item (b)} |
| Iron, Fe | 510 ± 23 | 530 ± 0 |
| Manganese, Mn | 430 ± 25 | 470 ± 6 |
| Strontium, Sr | 210 ± 6 | 230 ± 0 |
| Zinc, Zn | 3300 ± 58 | 3600 ± 58 |

Wound healing and nutrition are closely linked, where nutrient deficiency impedes the normal healing process by prolonging the inflammatory phase, and reduces fibroblast proliferation and collagen synthesis. Iron homeostasis in the skin is important for the cutaneous wound healing process, where both iron deficiency and overload have shown negative effects. Strontium salt has been shown to suppress TNF-levels and thus inflammation levels, while both zinc and manganese have been shown to modulate the expression of integrins affecting, for example, the proliferation phase of wound healing. Zinc supplements have been found to enhance wound healing, especially when administrated topically, where zinc ions stimulate epithelialization and reduce superinfections and the amount of necrotic material. Additionally, zinc has been found effective specifically for healing of diabetic foot ulcers.

Bioburden Analysis. Bioburden analysis was performed to determine the sterility of the finished product. The suitability of test method and growth promotion test was successful, since the small amount (<100 CFU) of micro-organisms inoculated onto agar was recovered. The inoculation together with either the HTX solution or the Collex patch showed no inhibitory (antimicrobial) effect. Both the HTX solution and the Collex patch were found to be sterile with no growth found in any of the samples, indicating adequate routines during aseptic formulation, production, and packaging.

Bioactivity and Biocompatibility. Collagens are converted from pro-collagens and play critical roles in wound healing. HTX stimulates the production of pro-collagen I alpha 1. For this reason, the bioactivity of released HTX was determined based on its ability to stimulate collagen production, measured by the procollagen I alpha 1 ELISA assay. Collex containing HTX resulted in significantly higher production of procollagen I alpha 1 compared to negative controls (p<0.01). The R4 ink printed control without HTX did not affect pro-collagen I alpha 1 production. A maintained bioactivity was confirmed for Collex after storage for 4 weeks in a fridge as well as at room temperature, see FIG. 8. In the release assay, only proteins were measured and the release of fatty acids and other compounds that may contribute to the effect of HTX were not detected. However, because results from the pro-collagen I alpha 1 assay showed that the increase in pro-collagen was similar for the Collex matrix.

### Example 6

### Production of Molded Collex Matrix

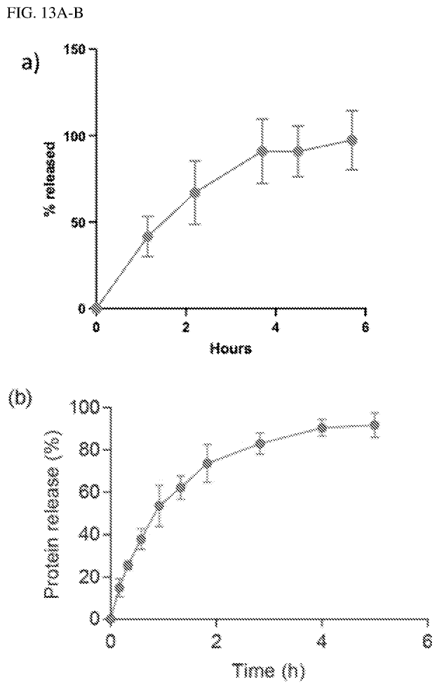

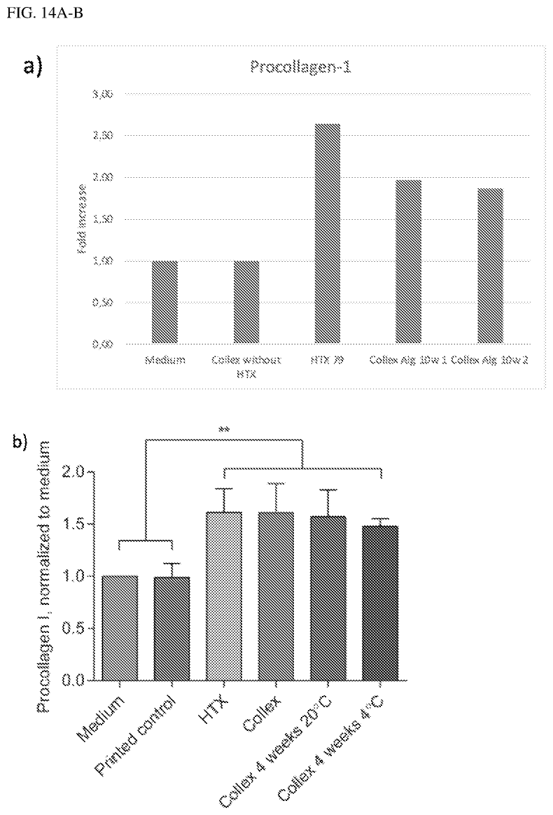

This example describes methods for the production of Collex wound healing matrices by molding. A mixture was made comprising 12% volume/weight (vw) HTX (heat-treated salmon egg extract comprising: Protein: 90-130 mg/ml; DNA: 1.4 mg/ml; pH: 6.5-7.5); 0.8% NaCl and 2.0% alginate (Pronova UP MVG; Viscosity [mPa*s]: >200; Appr. Mw [kDa]: >200; G/M Ratio: ≥1.5). The mixture was then transferred to a shallow rectangular mold. The mixture was then cross-linked by addition of 0.1% CaCl₂) and the resulting Collex sheet was removed from the mold. Protein release from the molded matrix was then compared to release from the 3D printed matrix described above. The data is provided in FIGS. 13 and 14. Release from the molded Collex matrix into an aqueous is similar to the 3D printed matrix containing nanocellulose. FIG. 13. Further, the increase in Procollagen-1 with the molded Collex matrix was similar to the 3D-printed Collex matrix with nanocellulose and the molded matrix is stable for at least 10 weeks at 4° C. (FIG. 14).

### Example 7

### Reduction of Reactive Oxygen Species (ROS)

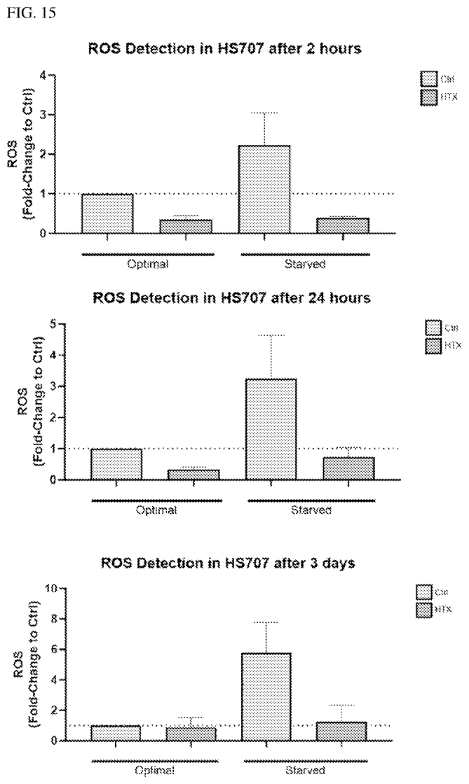

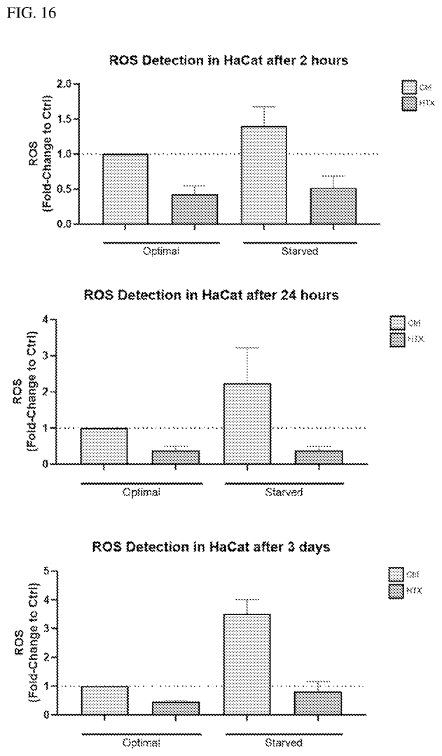

The example provides data showing that HTX reduces reaction oxygen species in fibroblast (Hs707) and keratinocyte (HaCaT) cells and thus exhibits an antioxidant activity. Briefly, HTX (5% v/v) was applied to fibroblast (Hs707) and keratinocyte (HaCaT) cells in vitro under conditions that induced cell stress. The data is presented in FIGS. 15 and 16. The antioxidant effect due to HTX application was rapid as ROS was already reduced after 2 hours HTX treatment. The antioxidant effect of HTX was transient as it was less pronounced after 3 days. Finally, ROS reduction is more pronounced in stressed cells (stressed by serum-starvation).

### REFERENCES

(1) Rowan, M. P.; Cancio, L. C.; Elster, E. A.; Burmeister, D. M.; Rose, L. F.; Natesan, S.; Chan, R. K.; Christy, R. J.; Chung, K. K. Burn Wound Healing and Treatment: Review and Advancements. Crit. Care Lond. Engl. 2015, 19, 243. https://doi.org/10.1186/s13054-015-0961-2.
(2) Ozgok Kangal, M. K.; Regan, J.-P. Wound Healing. In StatPearls; StatPearls Publishing: Treasure Island (FL), 2022.
(3) Lateef, Z.; Stuart, G.; Jones, N.; Mercer, A.; Fleming, S.; Wise, L. The Cutaneous Inflammatory Response to Thermal Burn Injury in a Murine Model. Int. J. Mol. Sci. 2019, 20 (3), E538. https://doi.org/10.3390/ijms20030538.
(4) Salibian, A. A.; Rosario, A. T. D.; Severo, L. D. A. M.; Nguyen, L.; Banyard, D. A.; Toranto, J. D.; Evans, G. R. D.; Widgerow, A. D. Current Concepts on Burn Wound Conversion-A Review of Recent Advances in Understanding the Secondary Progressions of Burns. Burns J. Int. Soc. Burn Inj. 2016, 42 (5), 1025-1035. https://doi.org/10.1016/j.burns.2015.11.007.
(5) Dunnill, C.; Patton, T.; Brennan, J.; Barrett, J.; Dryden, M.; Cooke, J.; Leaper, D.; Georgopoulos, N. T. Reactive Oxygen Species (ROS) and Wound Healing: The Functional Role of ROS and Emerging ROS-Modulating Technologies for Augmentation of the Healing Process. Int. Wound J. 2017, 14 (1), 89-96. https://doi.org/10.1111/iwj.12557.
(6) Han, Y. P.; Tuan, T. L.; Wu, H.; Hughes, M.; Garner, W. L. TNF-Alpha Stimulates Activation of pro-MMP2 in Human Skin through NF-(Kappa) B Mediated Induction of MT1-MMP. J. Cell Sci. 2001, 114 (Pt 1), 131-139. https://doi.org/10.1242/jcs.114.1.131.
(7) Parihar, A.; Parihar, M. S.; Milner, S.; Bhat, S. Oxidative Stress and Anti-Oxidative Mobilization in Burn Injury. Burns J. Int. Soc. Burn Inj. 2008, 34 (1), 6-17. https://doi.org/10.1016/j.burns.2007.04.009.
(8) Ogawa, R. Keloid and Hypertrophic Scars Are the Result of Chronic Inflammation in the Reticular Dermis. Int. J. Mol. Sci. 2017, 18 (3), E606. https://doi.org/10.3390/ijms18030606.
(9) Zhang, D.; Wang, B.; Sun, Y.; Wang, C.; Mukherjee, S.; Yang, C.; Chen, Y. Injectable Enzyme-Based Hydrogel Matrix with Precisely Oxidative Stress Defense for Promoting Dermal Repair of Burn Wound. Macromol. Biosci. 2020, 20 (6), e2000036. https://doi.org/10.1002/mabi.202000036.
(10) Skiftesvik, J.; Thormodsæter Fitjar, G.; Onarheim, H.; Ljones Brekke, R. Brannskader. In Mine Metodebφker; 2020.
(11) Aggarwala, S.; Harish, V.; Roberts, S.; Brady, M.; Lajevardi, S.; Doherty, J.; D'Souza, M.; Haertsch, P. A.; Maitz, P. K. M.; Issler-Fisher, A. C. Treatment of Partial Thickness Burns: A Prospective, Randomized Controlled Trial Comparing Four Routinely Used Burns Dressings in an Ambulatory Care Setting. J. Burn Care Res. Off. Publ. Am. Burn Assoc. 2021, 42 (5), 934-943. https://doi.org/10.1093/jbcr/iraa158.
(12) Yunoki, S.; Kohta, M.; Ohyabu, Y.; Iwasaki, T. In Vitro Parallel Evaluation of Antibacterial Activity and Cytotoxicity of Commercially Available Silver-Containing Wound Dressings. Plast. Surg. Nurs. Off. J. Am. Soc. Plast. Reconstr. Surg. Nurses 2015, 35 (4), 203-211. https://doi.org/10.1097/PSN.0000000000000096.
(13) Brouillard, C.; Bursztejn, A.-C.; Latarche, C.; Cuny, J.-F.; Truchetet, F.; Goulle, J.-P.; Schmutz, J.-L. Silver Absorption and Toxicity Evaluation of Silver Wound Dressings in 40 Patients with Chronic Wounds. J. Eur. Acad. Dermatol. Venereol. JEADV 2018, 32 (12), 2295-2299. https://doi.org/10.1111/jdv.15055.
(14) Comino-Sanz, I. M.; Lopez-Franco, M. D.; Castro, B.; Pancorbo-Hidalgo, P. L. The Role of Antioxidants on Wound Healing: A Review of the Current Evidence. J. Clin. Med. 2021, 10 (16), 3558. https://doi.org/10.3390/jcm10163558.
(15) Verdolino, D. V.; Thomason, H. A.; Fotticchia, A.; Cartmell, S. Wound Dressings: Curbing Inflammation in Chronic Wound Healing. Emerg. Top. Life Sci. 2021, 5 (4), 523-537. https://doi.org/10.1042/ETLS20200346.
(16) Tudoroiu, E.-E.; Dinu-Pirvu, C.-E.; Albu Kaya, M. G.; Popa, L.; Anuta, V.; Prisada, R. M.; Ghica, M. V. An Overview of Cellulose Derivatives-Based Dressings for Wound-Healing Management. Pharm. Basel Switz. 2021, 14 (12), 1215. https://doi.org/10.3390/ph14121215.
(17) Zhang, M.; Zhao, X. Alginate Hydrogel Dressings for Advanced Wound Management. Int. J. Biol. Macromol. 2020, 162, 1414-1428. https://doi.org/10.1016/j.ijbiomac.2020.07.311.
(18) Sun, C.; Zhang, S. Immune-Relevant and Antioxidant Activities of Vitellogenin and Yolk Proteins in Fish. Nutrients 2015, 7 (10), 8818-8829. https://doi.org/10.3390/nu7105432.
(19) Searle, T.; Ali, F. R.; Al-Niaimi, F. Zinc in Dermatology. J. Dermatol. Treat. 2022, 33 (5), 2455-2458. https://doi.org/10.1080/09546634.2022.2062282.
(20) Nicolaou, A. Eicosanoids in Skin Inflammation. Prostaglandins Leukot. Essent. Fatty Acids 2013, 88 (1), 131-138. https://doi.org/10.1016/j.plefa.2012.03.009.
(21) Michalak, M.; Pierzak, M.; Kręcisz, B.; Suliga, E. Bioactive Compounds for Skin Health: A Review. Nutrients 2021, 13 (1), 203. https://doi.org/10.3390/nu13010203.
(22) Baxter, E. W.; Graham, A. E.; Re, N. A.; Carr, I. M.; Robinson, J. I.; Mackie, S. L.; Morgan, A. W. Standardized Protocols for Differentiation of THP-1 Cells to Macrophages with Distinct M(IFNγ+LPS), M(IL-4) and M(IL-10) Phenotypes. J. Immunol. Methods 2020, 478, 112721. https://doi.org/10.1016/j.jim.2019.112721.
(23) Clemm, C.; Blom, K.; Heldrup, M.; Eriksson, G. L.; Andrys, D.; Bysell, H.; Lund, H. Salmon-Roe Derived Biologic Actives Accelerate Wound Healing in Burns Inflicted in Human Explanted Skin; 2015.
(24) Rutala, W. A.; Weber, D. J. Disinfection and Sterilization in Health Care Facilities: An Overview and Current Issues. Infect. Dis. Clin. North Am. 2016, 30 (3), 609-637. https://doi.org/10.1016/j.idc.2016.04.002.
(25) Schneier, M.; Razdan, S.; Miller, A. M.; Briceno, M. E.; Barua, S. Current Technologies to Endotoxin Detection and Removal for Biopharmaceutical Purification. Biotechnol. Bioeng. 2020, 117 (8), 2588-2609. https://doi.org/10.1002/bit.27362.
(26) Shapouri-Moghaddam, A.; Mohammadian, S.; Vazini, H.; Taghadosi, M.; Esmaeili, S.-A.; Mardani, F.; Seifi, B.; Mohammadi, A.; Afshari, J. T.; Sahebkar, A. Macrophage Plasticity, Polarization, and Function in Health and Disease. J. Cell. Physiol. 2018, 233 (9), 6425-6440. https://doi.org/10.1002/jcp.26429.
(27) O'Brien, E. M.; Spiller, K. L. Pro-Inflammatory Polarization Primes Macrophages to Transition into a Distinct M2-like Phenotype in Response to IL-4. J. Leukoc. Biol. 2022, 111 (5), 989-1000. https://doi.org/10.1002/JLB.3A0520-338R.
(28) Junttila, I. S. Tuning the Cytokine Responses: An Update on Interleukin (IL)-4 and IL-13 Receptor Complexes. Front. Immunol. 2018, 9, 888. https://doi.org/10.3389/fimmu.2018.00888.
(29) Rendra, E.; Riabov, V.; Mossel, D. M.; Sevastyanova, T.; Harmsen, M. C.; Kzhyshkowska, J. Reactive Oxygen Species (ROS) in Macrophage Activation and Function in Diabetes. Immunobiology 2019, 224 (2), 242-253. https://doi.org/10.1016/j.imbio.2018.11.010.
(30) Matylevitch, N. P.; Schuschereba, S. T.; Mata, J. R.; Gilligan, G. R.; Lawlor, D. F.; Goodwin, C. W.; Bowman, P. D. Apoptosis and Accidental Cell Death in Cultured Human Keratinocytes after Thermal Injury. Am. J. Pathol. 1998, 153 (2), 567-577. https://doi.org/10.1016/S0002-9440 (10) 65599-X.
(31) Iwasaki, K.; Izawa, M.; Mihara, M. Thermal Injury Induces Both Necrosis and Apoptosis in Rat Skin. Br. J. Dermatol. 1997, 137 (4), 647-648. https://doi.org/10.1111/j.1365-2133.1997.tb03805.x.
(32) Nuutila, K.; Eriksson, E. Moist Wound Healing with Commonly Available Dressings. Adv. Wound Care 2021, 10 (12), 685-698. https://doi.org/10.1089/wound.2020.1232.
(33) Fonder, M. A.; Mamelak, A. J.; Lazarus, G. S.; Chanmugam, A. Occlusive Wound Dressings in Emergency Medicine and Acute Care. Emerg. Med. Clin. North Am. 2007, 25 (1), 235-242. https://doi.org/10.1016/j.emc.2007.01.012.
(34) El Ayadi, A.; Jay, J. W.; Prasai, A. Current Approaches Targeting the Wound Healing Phases to Attenuate Fibrosis and Scarring. Int. J. Mol. Sci. 2020, 21 (3), E1105. https://doi.org/10.3390/ijms21031105.
(35) Carney, B. C.; Chen, J. H.; Kent, R. A.; Rummani, M.; Alkhalil, A.; Moffatt, L. T.; Rosenthal, D. S.; Shupp, J. W. Reactive Oxygen Species Scavenging Potential Contributes to Hypertrophic Scar Formation. J. Surg. Res. 2019, 244, 312-323. https://doi.org/10.1016/j.jss.2019.06.006.
(36) Wang, Z.-C.; Zhao, W.-Y.; Cao, Y.; Liu, Y.-Q.; Sun, Q.; Shi, P.; Cai, J.-Q.; Shen, X. Z.; Tan, W.-Q. The Roles of Inflammation in Keloid and Hypertrophic Scars. Front. Immunol. 2020, 11, 603187. https://doi.org/10.3389/fimmu.2020.603187.
(37) Dolgachev, V. A.; Ciotti, S.; Liechty, E.; Levi, B.; Wang, S. C.; Baker, J. R.; Hemmila, M. R. Dermal Nanoemulsion Treatment Reduces Burn Wound Conversion and Improves Skin Healing in a Porcine Model of Thermal Burn Injury. J. Burn Care Res. Off. Publ. Am. Burn Assoc. 2021, 42 (6), 1232-1242. https://doi.org/10.1093/jbcr/irab118.
(38) Kim, Y. E.; Kim, J. ROS-Scavenging Therapeutic Hydrogels for Modulation of the Inflammatory Response. ACS Appl. Mater. Interfaces 2021. https://doi.org/10.1021/acsami.1c18261.
(39) Thi, P. L.; Lee, Y.; Tran, D. L.; Thi, T. T. H.; Kang, J. I.; Park, K. M.; Park, K. D. In Situ Forming and Reactive Oxygen Species-Scavenging Gelatin Hydrogels for Enhancing Wound Healing Efficacy. Acta Biomater. 2020, 103, 142-152. https://doi.org/10.1016/j.actbio.2019.12.009.
(40) Calder, P. C. Marine Omega-3 Fatty Acids and Inflammatory Processes: Effects, Mechanisms and Clinical Relevance. Biochim. Biophys. Acta 2015, 1851 (4), 469-484. https://doi.org/10.1016/j.bbalip.2014.08.010.
(41) Djuricic, I.; Calder, P. C. Beneficial Outcomes of Omega-6 and Omega-3 Polyunsaturated Fatty Acids on Human Health: An Update for 2021. Nutrients 2021, 13 (7), 2421. https://doi.org/10.3390/nu13072421.
(42) Jarosz, M.; Olbert, M.; Wyszogrodzka, G.; Młyniec, K.; Librowski, T. Antioxidant and Anti-Inflammatory Effects of Zinc. Zinc-Dependent NF-KB Signaling. Inflammopharmacology 2017, 25 (1), 11-24. https://doi.org/10.1007/s10787-017-0309-4.
(43) Lansdown, A. B. G.; Mirastschijski, U.; Stubbs, N.; Scanlon, E.; Agren, M. S. Zinc in Wound Healing: Theoretical, Experimental, and Clinical Aspects. Wound Repair Regen. Off. Publ. Wound Heal. Soc. Eur. Tissue Repair Soc. 2007, 15 (1), 2-16. https://doi.org/10.1111/j.1524-475X.2006.00179.x.
(44) Wessels, I.; Maywald, M.; Rink, L. Zinc as a Gatekeeper of Immune Function. Nutrients 2017, 9 (12), E1286. https://doi.org/10.3390/nu9121286.
(45) Ågren, M. S.; Phothong, N.; Burian, E. A.; Mogensen, M.; Hædersdal, M.; Jorgensen, L. N. Topical Zinc Oxide Assessed in Two Human Wound-Healing Models. Acta Derm. Venereol. 2021, 101 (5), adv00465. https://doi.org/10.2340/00015555-3829.
(46) Deng, L.; Du, C.; Song, P.; Chen, T.; Rui, S.; Armstrong, D. G.; Deng, W. The Role of Oxidative Stress and Antioxidants in Diabetic Wound Healing. Oxid. Med. Cell. Longev. 2021, 2021, 8852759. https://doi.org/10.1155/2021/8852759.
(47) Bosanquet, D. C.; Harding, K. G. Wound Duration and Healing Rates: Cause or Effect? Wound Repair Regen. Off. Publ. Wound Heal. Soc. Eur. Tissue Repair Soc. 2014, 22 (2), 143-150. https://doi.org/10.1111/wrr.12149.
(48) Stenlund et al., Development of an All-Marine 3D Printed Bioactive Hydrogel Dressing for Treatment of Hard-to-Heal Wounds Polymers 2023, 15, 2627.

All publications and patents mentioned in the above specification are herein incorporated by reference. Various modifications and variations of the described method of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in medicine, medicinal chemistry, organic chemistry, virology, biology, genetics, or related fields are intended to be within the scope of the following claims.

What is claimed is:

1. An article comprising a matrix formed from at least a first polysaccharide, said matrix further comprising a differentiable cell extract and wherein the first polysaccharide is from a source different from the differentiable cell extract.

2. The article of claim 1, wherein the first polysaccharide is alginate.

3. The article of claim 2, wherein the weight/weight percent of the alginate in the article is from 1.0% to 10.0%.

4. The article of any one of claims 1 to 3, further comprising a second polysaccharide from a source different from the differentiable cell extract.

5. The article of claim 4, wherein the second polysaccharide is nanocellulose.

6. The article of claim 5, wherein the weight/weight percent of the nanocellulose in the article is from 1.0% to 10.0%.

7. The article of any one of claims 1 to 6, wherein the nanocellulose is supplemented with mannitol.

8. The article of claim 6, wherein the weight/weight percent of the mannitol used to supplement the nanocellulose is from 1.0% to 10.0%.

9. The article of any one of claims 1 to 8, wherein the volume/weight percent of the differentiable cell extract in the article is from 5.0% to 20.0%.

10. The article of anyone of claims 1 to 9, wherein the differentiable cell extract is a fish egg extract.

11. The article of claim 10, wherein the fish egg extract is a salmonid egg extract.

12. The article any one of claims 10 to 11, wherein the fish egg extract is an unfertilized egg extract.

13. The article of any one of claims 10 to 12, wherein the fish egg extract is characterized in having one or more of properties (a) to (f):
a) from 10 to 500 mg/ml protein and most preferably from 50 to 200 mg/ml protein, in an aqueous solution;
b) from 0.1 to 10 mg/ml RNA;
c) from 0.1 to 10 mg/ml DNA;
d) from 0.1-10% lipids w/w
e) an osmolarity of from 200 to 600 mOsm, most preferably from 330 to 440 mOsm; and
f) a pH of from about 5.0 to 7.7.

14. The article of claim 13, wherein the fish egg extract has properties: (a) and (b); (a) and (c); (a) and (d); (a) and (e); (a) and (f); (a), (b), and (c); (a), (b), and (d); (a), (b), and (e); (a), (b), and (f); (a), (c), and (d); (a), (c), and (e); (a), (c), and (f); (a), (d), and (e); (a), (d), and (f); (a), (b), (c), and (d); (a), (b), (c), and (e); (a), (b), (c), and (f); (a), (c), (d), and (e); (a), (c), (d), and (f); (a), (c), (e), and (f); (a), (b), (c), (d) and (e); (a), (b), (c), (d) and (f); (a), (c), (d), (e) and (f); or (a), (b), (c), (d), (e), and (f).

15. The article any one of claims 10 to 14, wherein the fish egg extract is a heat-treated fish egg extract.

16. The article of claim 15, wherein the heat-treated fish egg extract is prepared by heating the fish egg extract to from 90 to 100 degrees Celsius for from 1 to 30 minutes.

17. The article of any one of claims 1 to 16, wherein the first or the first and second polysaccharides are cross-linked.

18. The article of any one of claims 1 to 17, wherein the matrix is a gel matrix.

19. The article of any one of claims 1 to 18, wherein the matrix is formed in a grid pattern.

20. An article comprising a matrix formed from alginate, said matrix further comprising a heat-treated Salmo salar egg extract, wherein the weight/weight percent of the alginate in the article is from 1.0% to 10.0% and the volume/weight percent of the heat-treated Salmo salar egg extract in the article is from 5.0% to 20.0%.

21. The article of claim 20, further comprising nanocellulose.

22. The article of claim 21, wherein the weight/weight percent of nanocellulose in the article is from 1.0% to 10.0%.

23. The article of any one of claims 1 to 6, wherein the nanocellulose is supplemented with mannitol.

24. The article of claim 6, wherein the weight/weight percent of the mannitol used to supplement the nanocellulose is from 1.0% to 10.0%.

25. The article any one of claims 20 to 24, wherein the heat-treated Salmo salar egg extract is prepared from unfertilized eggs.

26. The article of any one of claims 20 to 25, wherein the heat-treated Salmo salar egg extract is characterized in having one or more of properties (a) to (f):
a) from 50 to 500 mg/ml protein and most preferably from 10 to 5000 mg/ml protein and most preferably from 50 to 200 mg/ml protein, in an aqueous solution;
b) from 0.1 to 10 mg/ml RNA;
c) from 0.1 to 10 mg/ml DNA;
d) from 0.1-10% lipids w/w
e) an osmolarity of from 200 to 600 mOsm, most preferably from 330 to 440 mOsm; and
f) a pH of from about 5.0 to 7.7.

27. The article of claim 26, wherein the fish egg extract has properties: (a) and (b); (a) and (c); (a) and (d); (a) and (e); (a) and (f); (a), (b), and (c); (a), (b), and (d); (a), (b), and (e); (a), (b), and (f); (a), (c), and (d); (a), (c), and (e); (a), (c), and (f); (a), (d), and (e); (a), (d), and (f); (a), (b), (c), and (d); (a), (b), (c), and (e); (a), (b), (c), and (f); (a), (c), (d), and (e); (a), (c), (d), and (f); (a), (c), (e), and (f); (a), (b), (c), (d) and (e); (a), (b), (c), (d) and (f); (a), (c), (d), (e) and (f); or (a), (b), (c), (d), (e), and (f).

28. The article of any one of claims 20 to 27, wherein the heat-treated Salmo salar egg extract is prepared by heating the Salmo salar egg extract to from 90 to 100 degrees Celsius for from 1 to 30 minutes.

29. The article of any one of claims 20 to 28, wherein the matrix is a cross-linked gel matrix.

30. The article of any one of claims 20 to 29, wherein the matrix is formed in a grid pattern.

31. A method of producing a wound healing article comprising:
forming an aqueous mixture of at least a first polysaccharide and a fish egg extract;
forming a matrix from the aqueous mixture to provide the wound healing article; and
wherein the at least a first polysaccharide is from a source different from the fish egg extract.

32. The method of claim 31, further comprising the step of cross linking the matrix to provide the wound healing article.

33. The method of any one of claims 31 to 32, wherein the first polysaccharide is alginate.

34. The method of claim 33, wherein the alginate is included in the mixture at a weight/weight percent of from 1.0% to 10.0%.

35. The method of any one of claims 31 to 34, further comprising including a second polysaccharide in the aqueous mixture, wherein the second polysaccharide is from a source different from the fish egg extract.

36. The method of claim 35, wherein the second polysaccharide is nanocellulose.

37. The method of claim 36, wherein the nanocellulose is included in the mixture at a weight/weight percent of from 1.0% to 10.0%.

38. The method of any one of claims 31 to 37, wherein the fish egg extract is included in the mixture at a volume/weight percent of from 5.0% to 20.0%.

39. The method of any one of claims 31 to 38, wherein the fish egg extract is from unfertilized fish eggs.

40. The method of any one of claims 31 to 39, wherein the fish egg extract is a Salmo salar egg extract.

41. The method of any one of claims 31 to 40, wherein the fish egg extract is a heat-treated fish egg extract.

42. The method of claim 41, wherein the heat-treated fish egg extract is prepared by heating the fish egg extract to a temperature of from 90 to 100 degrees Celsius for from 1 to 30 minutes.

43. The method of any one of claims 40 to 42, wherein the heat-treated Salmo salar egg extract is characterized is characterized in having one or more of properties (a) to (f):
a) from 10 to 500 mg/ml protein and most preferably from 50 to 200 mg/ml protein, in an aqueous solution;
b) from 0.1 to 10 mg/ml RNA;
c) from 0.1 to 10 mg/ml DNA;
d) from 0.1-10% lipids w/w
e) an osmolarity of from 200 to 60 mOsm, most preferably from 330 to 440 mOsm; and
f) a pH of from about 5.0 to 7.7.

44. The method of claim 43, wherein the fish egg extract has properties: (a) and (b); (a) and (c); (a) and (d); (a) and (e); (a) and (f); (a), (b), and (c); (a), (b), and (d); (a), (b), and (e); (a), (b), and (f); (a), (c), and (d); (a), (c), and (e); (a), (c), and (f); (a), (d), and (e); (a), (d), and (f); (a), (b), (c), and (d); (a), (b), (c), and (e); (a), (b), (c), and (f); (a), (c), (d), and (e); (a), (c), (d), and (f); (a), (c), (e), and (f); (a), (b), (c), (d) and (e); (a), (b), (c), (d) and (f); (a), (c), (d), (e) and (f); or (a), (b), (c), (d), (e), and (f).

45. The method of any one of claims 31 to 44, wherein the nanocellulose is supplemented with mannitol.

46. The method of claim 45, wherein the weight/weight percent of the mannitol used to supplement the nanocellulose is from 1.0% to 10.0%.

47. The method of anyone of claims 31 to 46, wherein the aqueous mixture further comprises CaCl₂).

48. The method of claim 47, wherein the CaCl₂) is included in the aqueous mixture at a concentration of from 0.01 to 0.1 M.

49. The method of any one of claims 31 to 48, wherein the matrix is formed by printing the aqueous mixture onto a substrate.

50. The method of any one of claims 31 to 48, wherein the matrix is formed by molding the aqueous mixture.

51. The method of any one of claims 31 to 50, wherein the matrix is cross-linked by treating the matrix with a cross-linking solution comprising CaCl₂) at a concentration of from 0.01 to 0.1 M.

52. The method of claim 51, wherein the cross-linking solution further comprises a weight/weight percent of NaCl of from 0.5% to 1.5%.

53. The method of any one of claims 51 to 52, wherein the cross-linking solution further comprises the fish egg extract at a volume/weight percent of from 5.0% to 20.0%.

54. The method of any one of claims 31 to 53, wherein the matrix is a gel.

55. A matrix made by the method of any one of claims 31 to 54.

56. Article of any one of claims 1 to 30 or matrix of claim 55 for use in treating a wound in a subject.

57. Use of claim 56, wherein the wound is a burn wound.

58. Use of claim 56, wherein the wound is a chronic wound.

59. Use of any one of claims 56 to 58, wherein the article or solid matrix is topically applied to the wound.

60. Method of treating a wound in a subject in need thereof comprising applying an article of any one of claims 1 to 30 or solid matrix of claim 55 to the wound.

61. Method of claim 60, wherein the wound is a burn wound.

62. Method of claim 60, wherein the wound is a chronic wound.

63. Use of an article of any one of claims 1 to 30 or matrix of claim 55 to reduce reactive oxygen species in a subject in need thereof.

64. Use of claim 63, wherein the subject has a wound and the matrix is applied to the wound.

65. Use of claim 64, wherein the subject has skin inflammation and the matrix is applied to the site of skin inflammation.

66. Use of a fish egg extract or formulation thereof to reduce reactive oxygen species in a subject in need thereof, wherein the fish egg extract is characterized in having one or more of properties (a) to (f):
a) from 50 to 500 mg/ml protein and most preferably from 10 to 5000 mg/ml protein and most preferably from 50 to 200 mg/ml protein, in an aqueous solution;
b) from 0.1 to 10 mg/ml RNA;
c) from 0.1 to 10 mg/ml DNA;
d) from 0.1-10% lipids w/w
e) an osmolarity of from 200 to 600 mOsm, most preferably from 330 to 440 mOsm; and
f) a pH of from about 5.0 to 7.7

67. Use of claim 66, wherein the subject has a wound and the fish egg extract is applied to the wound.

68. Use of claim 66, wherein the subject has skin inflammation and the fish egg extract is applied to the site of skin inflammation.

69. Use of any one of claims 66 to 68, wherein the fish egg extract has properties: (a) and (b); (a) and (c); (a) and (d); (a) and (e); (a) and (f); (a), (b), and (c); (a), (b), and (d); (a), (b), and (e); (a), (b), and (f); (a), (c), and (d); (a), (c), and (e); (a), (c), and (f); (a), (d), and (e); (a), (d), and (f); (a), (b), (c), and (d); (a), (b), (c), and (e); (a), (b), (c), and (f); (a), (c), (d), and (e); (a), (c), (d), and (f); (a), (c), (e), and (f); (a), (b), (c), (d) and (e); (a), (b), (c), (d) and (f); (a), (c), (d), (e) and (f); or (a), (b), (c), (d), (e), and (f).

70. Use of any one of claims 66 to 69, wherein the fish egg extract is a heat-treated fish egg extract.

71. Use of any one of claims 66 to 70, wherein the fish egg extract is a heat-treated fish egg extract.
